(19) United States
(12) Patent Application Publication
(10) Pub. No.: US 2023/0279463 A1
(43) Pub. Date: Sep. 7, 2023

(54) Computationally Derived Minimum Inhibitory Concentration Prediction from Multi-Dimensional Flow Cytometric Susceptibility Testing

(71) Applicant: BECTON, DICKINSON AND COMPANY, Franklin Lakes, NJ (US)

(72) Inventors: WARREN PORTER, Chapel Hill, NC (US); FRANCES POYEN TONG, Carrboro, NC (US)

(21) Appl. No.: 18/023,658

(22) PCT Filed: Aug. 31, 2021
(86) PCT No.: PCT/US2021/048403

§ 371 Date: Feb. 27, 2023

Related U.S. Application Data
(60) Provisional application No. 63/074,953, filed on Sep. 4, 2020.

(51) Int. Cl. C12Q 1/18; G06N 3/04

(52) U.S. Cl. CPC C12Q 1/18; G06N 3/04

(57) ABSTRACT

Methods for estimating a minimum inhibitory concentration of an antibiotic for a bacterial species. Methods according to certain embodiments include obtaining cytometric data (e.g., flow cytometer data) for a plurality of test samples and a control sample for the antibiotic and bacterial species, computing distance values that reflect a measure of variation between one or more pairs of samples, and assigning a minimum inhibitory concentration based on the computed distance values. Systems for practicing the subject methods are also provided. Non-transitory computer readable storage media are also described.

### CROSS-REFERENCE

Pursuant to 35 U.S.C. § 119 (e), this application claims priority to the filing date of U.S. Provisional Pat. Application Serial No. 63/074,953 filed Sep. 4, 2020, the disclosure of which application is incorporated herein by reference in its entirety.

### INTRODUCTION

The characterization of the susceptibility of a species of bacteria to an antibiotic has become an important part of medical research and has clinical applications in the treatment of patients. Methods of analyzing the susceptibility of a species of bacteria to an antibiotic using cytometric data, such as via flow cytometry, have broad applications in the field of biological and medical research. Methods of estimating a minimum inhibitory concentration of an antibiotic for a bacterial species using cytometric data, such as via flow cytometry, have applications in the treatment of patients.

Flow cytometry is a technique used to characterize and often times sort biological material, such as cells of a blood sample or a sample comprising bacterial cells or particles of interest in another type of biological or chemical sample. A flow cytometer typically includes a sample reservoir for receiving a fluid sample, such as a blood sample or a sample comprising bacterial cells, and a sheath reservoir containing a sheath fluid. The flow cytometer transports the particles (including cells, such as bacterial cells) in the fluid sample as a cell stream to a flow cell, while also directing the sheath fluid to the flow cell. To characterize the components of the flow stream, the flow stream is irradiated with light. Variations in the materials in the flow stream, such as morphologies (including variations in the morphologies of bacterial cells resulting from exposure to antibiotic) or the presence of fluorescent labels, may cause variations in the observed light and these variations allow for characterization and separation. For example, particles, such as molecules, analyte-bound beads, or individual cells, in a fluid suspension are passed by a detection region in which the particles are exposed to an excitation light, typically from one or more lasers, and the light scattering and fluorescence properties of the particles are measured. Particles or components thereof typically are labeled with fluorescent dyes to facilitate detection. A multiplicity of different particles or components may be simultaneously detected by using spectrally distinct fluorescent dyes to label the different particles or components. In some implementations, a multiplicity of photodetectors, one for each of the scatter parameters to be measured, and one or more for each of the distinct dyes to be detected are included in the analyzer. For example, some embodiments include spectral configurations where more than one sensor or detector is used per dye. The data obtained comprise the signals measured for each of the light scatter detectors and the fluorescence emissions.

Particle analyzers may further comprise means for recording the measured data and analyzing the data. For example, data storage and analysis may be carried out using a computer connected to the detection electronics. For example, the data can be stored in tabular form, where each row corresponds to data for one particle, and the columns correspond to each of the measured features. The use of standard file formats, such as an “FCS” file format, for storing data from a particle analyzer facilitates analyzing data using separate programs and/or machines. Using current analysis methods, the data typically are displayed in 1-dimensional histograms or 2-dimensional (2D) plots for ease of visualization, but other methods may be used to visualize multidimensional data.

The parameters measured using, for example, a flow cytometer, typically include light at the excitation wavelength scattered by the particle in a narrow angle along a mostly forward direction, referred to as forward scatter (FSC), the excitation light that is scattered by the particle in an orthogonal direction to the excitation laser, referred to as side scatter (SSC), and the light emitted from fluorescent molecules in one or more detectors that measure signal over a range of spectral wavelengths, or by the fluorescent dye that is primarily detected in that specific detector or array of detectors. Different cell types, different cell morphologies, or cells that differ based on whether they are alive or not, can be identified by their light scatter characteristics and fluorescence emissions resulting from labeling various cell proteins or other constituents with fluorescent dye-labeled antibodies or other fluorescent probes.

Both flow and scanning cytometers are commercially available from, for example, BD Biosciences (San Jose, Calif.). Flow cytometry is described in, for example, Landy et al. (eds.), Clinical Flow Cytometry, Annals of the New York Academy of Sciences Volume 677 (1993); Bauer et al. (eds.), Clinical Flow Cytometry: Principles and Applications, Williams & Wilkins (1993); Ormerod (ed.), Flow Cytometry: A Practical Approach, Oxford Univ. Press (1994); Jaroszeski et al. (eds.), Flow Cytometry Protocols, Methods in Molecular Biology No. 91, Humana Press (1997); and Shapiro, Practical Flow Cytometry, 4th ed., Wiley-Liss (2003); all incorporated herein by reference. Fluorescence imaging microscopy is described in, for example, Pawley (ed.), Handbook of Biological Confocal Microscopy, 2nd Edition, Plenum Press (1989), incorporated herein by reference.

The data obtained from an analysis of cells (or other particles) by flow cytometry are multidimensional when each cell corresponds to a point in a multidimensional space defined by the parameters measured. Populations of cells or particles are identified as clusters of points in the data space. The identification of clusters and, thereby, populations can be carried out manually by drawing a gate around a population displayed in one or more 2-dimensional plots, referred to as “scatter plots” or “dot plots,” of the data. Alternatively, population clusters can be identified, and gates that define the limits of the populations, can be determined automatically. Examples of methods for automated gating have been described in, for example, U.S. Pat. Nos. 4,845,653; 5,627,040; 5,739,000; 5,795,727; 5,962,238; 6,014,904; and 6,944,338; and U.S. Pat. Pub. No. 2012/0245889, each incorporated herein by reference.

Characterizing the effectiveness of an antibiotic with respect to a bacterial species, can present a challenge insofar as it can be quite time consuming. For example, culture-based methods for antibiotic susceptibility testing may in some cases take two to three days to generate results. Utilizing cytometric data to facilitate characterizing the effectiveness of an antibiotic with respect to a bacterial species can return actionable results in a much shorter time span.

However, utilizing the different characteristics of analytes (e.g., bacterial cells treated with antibiotics) to characterize the effectiveness of an antibiotic with respect to a bacterial species often presents a further challenge because it may not be obvious how to determine such effectiveness in a way that is both consistent and rigorous. In particular, conventional methods of estimating the minimum inhibitory concentration (dosage) of an antibiotic with respect to a bacterial species often include subjective determinations. For example, estimating a minimum inhibitory concentration of an antibiotic with respect to a bacterial species may entail subjective determinations for a gating strategy to isolate subpopulations of the bacterial species for analysis. In other cases, estimating a minimum inhibitory concentration of an antibiotic with respect to a bacterial species may entail subjective determinations around choosing conditions and parameter values for ascertaining whether exposure to an antibiotic has resulted in changes in morphology of bacterial cells, an indicator of the susceptibility of a bacterial species to an antibiotic.

### SUMMARY

Aspects of the invention include methods for estimating a minimum inhibitory concentration of an antibiotic for a bacterial species. Methods according to certain embodiments include obtaining cytometric data for a plurality of test samples and a control sample for the antibiotic and bacterial species, computing distance values that reflect a measure of variation between one or more pairs of samples, and assigning a minimum inhibitory concentration based on the computed distance values. Systems for practicing the subject methods are also provided. Non-transitory computer readable storage media are also described.

In embodiments, assigning a minimum inhibitory concentration based on the computed distance values comprises fitting a curve to a plot of the distance values of the plurality of samples versus corresponding antibiotic concentrations of the plurality of samples, and assigning a minimum inhibitory concentration based on the fitted curve. In such embodiments, the computed distance values may be based on probability binning. In some embodiments, the probability binning may be based on a chi-squared statistic.

In certain embodiments, the computed distance values based on probability binning comprise setting ranges of cytometric data detected from cells in the control sample to a plurality of bins so that nearly equal numbers of cells in the control sample can be assigned to each bin in the plurality of bins, assigning cells in one of the test samples to the plurality of bins based on cytometric data detected from cells in the test sample, and computing a distance between the test sample and the control sample based on the cells in the test sample assigned to each bin. In instances, the computed distance values may be based on a T statistic.

In some embodiments, the curve fitted to the plot of the distance values of the plurality of samples versus corresponding antibiotic concentrations of the plurality of samples may be a logistic curve. In some cases, the lower horizontal asymptote of the fitted logistic curve is assigned a distance of zero. In some cases, the upper horizontal asymptote of the fitted logistic curve represents concentrations of the antibiotic at which substantially the entire sample is affected by the antibiotic.

In some examples, a minimum inhibitory concentration is assigned the antibiotic concentration corresponding to a point at which the slope of the logistic curve is maximum. In other examples, a minimum inhibitory concentration is assigned the antibiotic concentration corresponding to a point which is halfway between the upper and lower horizontal asymptotes of the logistic curve. In still other examples, a minimum inhibitory concentration is assigned the antibiotic concentration corresponding to a point that is a reliable detection limit of the curve.

In some embodiments, computing distance values between one or more pairs of samples comprises assigning cells of each sample to clusters of cell populations based on cytometric data from cells in each sample, matching clusters of cell populations from each sample with corresponding clusters of cell populations from one or more other samples, computing distance values between corresponding clusters of cell populations from pairs of samples based on cytometric data from cells in each cluster, and computing distance values between samples based on distance values between corresponding clusters of each sample. In some cases, assigning cells of each sample to clusters of cell populations comprises applying k-means clustering. In other cases, assigning cells of each sample to clusters of cell populations comprises applying a Self-Organizing Map. In certain embodiments, matching corresponding clusters of cell populations from each sample comprises applying a mixed edge cover algorithm.

In such embodiments, computing distances between corresponding clusters may be based on distribution parameters of each cluster. In instances, the distance values between corresponding clusters are computed using a Euclidean distance measurement. In other instances, the distance values between corresponding clusters are computed using a Mahalanobis distance measurement.

Some embodiments further comprise assigning each sample to a branch of a hierarchical tree based on distance values between samples. In some cases, the method further comprises assigning samples to groups based on distances between samples. In such cases, a minimum inhibitory concentration may be the antibiotic concentration corresponding to the sample with the lowest antibiotic concentration in a first group of samples that is the furthest distance away from a second group of samples, wherein the second group of samples includes the untreated control sample.

In some embodiments, a susceptibility or resistance of the antibiotic for the bacterial species is determined based on the minimum inhibitory concentration.

In some instances, methods according to the present disclosure further comprise preparing the plurality of test samples and the control sample. In such instances, preparing the plurality of test samples and the control sample may comprise treating a plurality of samples comprising bacterial cells of the bacterial species with the antibiotic at a plurality of different concentrations of the antibiotic and the control sample is not treated with the antibiotic.

In embodiments, the cytometric data is multi-parametric cytometry data. In some embodiments, the cytometric data comprises light scatter or marker data or a combination thereof. In such embodiments, the light scatter data may comprise forward scattered light or side scattered light or a combination thereof. In some examples, the marker data comprises fluorescent light emission data. In some cases, the fluorescent light emission data comprises frequency-encoded fluorescence data from cells.

In other embodiments, obtaining cytometric data from the plurality of test samples and the control sample comprises flow cytometrically analyzing the plurality of test samples and control sample.

Aspects of the present disclosure also include systems for estimating a minimum inhibitory concentration of an antibiotic for a bacterial species. In some embodiments, systems according to the present disclosure comprise an apparatus configured to obtain cytometric data for a plurality of test samples and a control sample for the antibiotic and bacterial species, a processor comprising memory operably coupled to the processor, wherein the memory comprises instructions stored thereon, which, when executed by the processor, cause the processor to: compute distance values that reflect a measure of variation between one or more pairs of samples, and assign a minimum inhibitory concentration based on the computed distance values.

In some embodiments, the memory comprises further instructions stored thereon, which, when executed by the processor, cause the processor to assign a minimum inhibitory concentration based on the computed distance values by: fitting a curve to a plot of the distance values of the plurality of samples versus corresponding antibiotic concentrations of the plurality of samples, and assigning a minimum inhibitory concentration based on the fitted curve.

In instances of systems according to the present disclosure, the computed distance values are based on probability binning. In such cases, probability binning may be based on a chi-squared statistic. In embodiments of systems, the memory comprises further instructions stored thereon, which, when executed by the processor, cause the processor to compute distance values based on probability binning by: setting ranges of cytometric data detected from cells in the control sample to a plurality of bins so that nearly equal numbers of cells in the control sample can be assigned to each bin in the plurality of bins, assigning cells in one of the test samples to the plurality of bins based on cytometric data detected from cells in the test sample, and computing a distance between the test sample and the control sample based on the cells in the test sample assigned to each bin. In other instances, the computed distance values are based on a T statistic.

In embodiments of the subject systems, the curve fitted to the plot of the distance values of the plurality of samples versus corresponding antibiotic concentrations of the plurality of samples is a logistic curve. In some examples, the lower horizontal asymptote of the fitted logistic curve is assigned a distance of zero. In some examples, the upper horizontal asymptote of the fitted logistic curve represents concentrations of the antibiotic at which substantially the entire sample is affected by the antibiotic.

In some instances, the memory comprises further instructions stored thereon, which, when executed by the processor, cause the processor to assign a minimum inhibitory concentration to be the antibiotic concentration corresponding to a point at which the slope of the logistic curve is maximum. In other instances, the memory comprises further instructions stored thereon, which, when executed by the processor, cause the processor to assign a minimum inhibitory concentration to be the antibiotic concentration corresponding to a point which is halfway between the upper and lower horizontal asymptotes of the logistic curve. In still other instances, the memory comprises further instructions stored thereon, which, when executed by the processor, cause the processor to assign a minimum inhibitory concentration the antibiotic concentration corresponding to a point that is a reliable detection limit of the curve.

In some embodiments of systems according to the present disclosure, the memory comprises further instructions stored thereon, which, when executed by the processor, cause the processor to compute distance values between one or more pairs of samples by: assigning cells of each sample to clusters of cell populations based on cytometric data from cells in each sample, matching clusters of cell populations from each sample with corresponding clusters of cell populations from one or more other samples, computing distance values between corresponding clusters of cell populations from pairs of samples based on cytometric data from cells in each cluster, and computing distance values between samples based on distance values between corresponding clusters of each sample.

In some examples of systems according to the present disclosure, assigning cells of each sample to clusters of cell populations comprises applying k-means clustering. In other examples, assigning cells of each sample to clusters of cell populations comprises applying a Self-Organizing Map.

In some embodiments of systems of interest, matching corresponding clusters of cell populations from each sample comprises applying a mixed edge cover algorithm.

In some instances of systems according to the present disclosure, computing distances between corresponding clusters is based on distribution parameters of each cluster. In certain instances, computing distances between corresponding clusters comprises measuring a distance between a cluster from a first test sample and a corresponding cluster from each other test sample and the control sample. In some cases, the distance values between corresponding clusters are computed using a Euclidean distance measurement. In other cases, the distance values between corresponding clusters are computed using a Mahalanobis distance measurement.

In some embodiments of systems of interest, the memory comprises further instructions stored thereon, which, when executed by the processor, cause the processor to assign each sample to a branch of a hierarchical tree based on distance values between samples. In other embodiments, the memory comprises further instructions stored thereon, which, when executed by the processor, cause the processor to assign samples to groups based on distances between samples. In such instances, a minimum inhibitory concentration is the antibiotic concentration corresponding to the sample with the lowest antibiotic concentration in a first group of samples that is the furthest distance away from a second group of samples, wherein the second group of samples includes the untreated control sample.

In certain embodiments of systems according to the present disclosure, a susceptibility or resistance of the antibiotic for the bacterial species is determined based on the minimum inhibitory concentration.

In instances of systems of interest, the cytometric data is multi-parametric cytometry data. In such instances, the cytometric data may comprise light scatter or marker data or a combination thereof. In some examples, the marker data comprises fluorescent light emission data. In other examples, the fluorescent light emission data comprises frequency-encoded fluorescence data from cells.

In some embodiments of systems, the apparatus is configured to obtain the cytometric data by analyzing the plurality of test samples and the control sample for the antibiotic and bacterial species. In other embodiments, the apparatus is configured to obtain cytometric data from the plurality of test samples and the control sample by flow cytometrically analyzing the plurality of test samples and control sample.

Aspects of the present disclosure also include a non-transitory computer readable storage medium for estimating a minimum inhibitory concentration of an antibiotic for a bacterial species. Non-transitory computer readable storage mediums according to certain embodiments include instructions stored thereon comprising algorithm for obtaining cytometric data for a plurality of test samples and a control sample for the antibiotic and bacterial species, algorithm for computing distance values that reflect a measure of variation between one or more pairs of samples, and algorithm for assigning a minimum inhibitory concentration based on the computed distance values. Non-transitory computer readable storage mediums according to certain embodiments may also include instructions stored thereon for assigning a minimum inhibitory concentration based on the computed distance values by: fitting a curve to a plot of the distance values of the plurality of samples versus corresponding antibiotic concentrations of the plurality of samples, and assigning a minimum inhibitory concentration based on the fitted curve.

In some embodiments of non-transitory computer readable storage mediums of interest, the computed distance values are based on probability binning. In such embodiments, the probability binning may be based on a chi-squared statistic. In some cases, non-transitory computer readable storage mediums further comprise instructions stored thereon for computing distance values based on probability binning by: setting ranges of cytometric data detected from cells in the control sample to a plurality of bins so that nearly equal numbers of cells in the control sample can be assigned to each bin in the plurality of bins, assigning cells in one of the test samples to the plurality of bins based on cytometric data detected from cells in the test sample, and computing a distance between the test sample and the control sample based on the cells in the test sample assigned to each bin. In some instances, the computed distance values are based on a T statistic.

In some embodiments, the curve fitted to the plot of the distance values of the plurality of samples versus corresponding antibiotic concentrations of the plurality of samples is a logistic curve. In examples, the lower horizontal asymptote of the fitted logistic curve may be assigned a distance of zero. In examples, the upper horizontal asymptote of the fitted logistic curve represents concentrations of the antibiotic at which substantially the entire sample is affected by the antibiotic.

In some embodiments of non-transitory computer readable storage mediums according to the present disclosure, a minimum inhibitory concentration is assigned to be the antibiotic concentration corresponding to a point at which the slope of the logistic curve is maximum. In some cases, a minimum inhibitory concentration is assigned to be the antibiotic concentration corresponding to a point which is halfway between the upper and lower horizontal asymptotes of the logistic curve. In some instances, a minimum inhibitory concentration is assigned to be the antibiotic concentration corresponding to a point that is a reliable detection limit of the curve.

Some embodiments of a non-transitory computer readable storage mediums according to the present disclosure further comprise instructions stored thereon for computing distance values between one or more pairs of samples by: assigning cells of each sample to clusters of cell populations based on cytometric data from cells in each sample, matching clusters of cell populations from each sample with corresponding clusters of cell populations from one or more other samples, computing distance values between corresponding clusters of cell populations from pairs of samples based on cytometric data from cells in each cluster, and computing distance values between samples based on distance values between corresponding clusters of each sample.

In some instances, assigning cells of each sample to clusters of cell populations comprises applying k-means clustering. In other instances, assigning cells of each sample to clusters of cell populations comprises applying a Self-Organizing Map. In some examples, matching corresponding clusters of cell populations from each sample comprises applying a mixed edge cover algorithm.

In some embodiments, computing distances between corresponding clusters is based on distribution parameters of each cluster. In some instances, computing distances between corresponding clusters comprises measuring a distance between a cluster from a first test sample and a corresponding cluster from each other test sample and the control sample. In some examples, the distance values between corresponding clusters are computed using a Euclidean distance measurement. In other examples, the distance values between corresponding clusters are computed using a Mahalanobis distance measurement.

Some embodiments of non-transitory computer readable storage mediums according to the present disclosure further comprise instructions stored thereon for assigning each sample to a branch of a hierarchical tree based on distance values between samples. Some embodiments of non-transitory computer readable storage mediums further comprise instructions stored thereon for assigning samples to groups based on distances between samples. In such embodiments, a minimum inhibitory concentration may be the antibiotic concentration corresponding to the sample with the lowest antibiotic concentration in a first group of samples that is the furthest distance away from a second group of samples, wherein the second group of samples includes the untreated control sample.

In some instances, a susceptibility or resistance of the antibiotic for the bacterial species is determined based on the minimum inhibitory concentration.

In some cases, the cytometric data is multi-parametric cytometry data. In some examples, the cytometric data comprises light scatter or marker data or a combination thereof. In such examples, the light scatter data may comprise forward scattered light or side scattered light or a combination thereof. In some embodiments, the marker data comprises fluorescent light emission data. In such embodiments, the fluorescent light emission data may comprise frequency-encoded fluorescence data from cells.

In embodiments, the subject methods, systems and non-transitory computer readable storage media are configured to analyze and/or process the data within a software or an analysis tool for analyzing and/or processing flow cytometer data, such as FlowJo®. The instant methods, systems and non-transitory computer readable storage media, or a portion thereof, can be implemented as software components of a software for analyzing data, such as FlowJo®. In these embodiments the subject methods, systems and non-transitory computer readable storage media according to the instant disclosure may function as a software “plugin” for an existing software package, such as FlowJo®.

Embodiments of the invention solve the problem of objectively and automatically quantifying effects of an antibiotic on a bacterial species based on cytometric data. That is, embodiments of the invention do not rely on subjective decisions regarding cytometric data, for example gating populations of the cytometric data for comparison or choosing conditions that appear different in scatter plots of the cytometric data. Embodiments of the invention facilitate making reproduceable estimates of a minimum inhibitory concentration of an antibiotic with respect to a bacterial species. Embodiments of the invention also facilitate making reproduceable estimates of a susceptibility or resistance of an antibiotic with respect to a bacterial species.

### BRIEF DESCRIPTION OF THE FIGURES

The invention may be best understood from the following detailed description when read in conjunction with the accompanying drawings. Included in the drawings are the following figures:

FIG. 1 depicts a flowchart that schematically demonstrates one exemplary instance of the subject method for determining a minimum inhibitory concentration based on cytometric data.

FIG. 2 depicts a flowchart that schematically demonstrates another exemplary instance of the subject method for estimating a minimum inhibitory concentration based on cytometric data.

FIG. 3 depicts an example of assigning a minimum inhibitory concentration for a bacterial species and antibiotic pair based on a fitted logistic curve according to embodiments of the subject method.

FIG. 4 presents a flowchart that schematically demonstrates one exemplary instance of the subject method for determining a minimum inhibitory concentration.

FIG. 5 depicts an example of assigning a minimum inhibitory concentration for an antibiotic-bacterial species pair based on characteristics of groups of test samples, as visualized on a hierarchical tree.

FIG. 6 depicts a flow cytometer according to certain embodiments.

FIG. 7 depicts a functional block diagram for one example of a processor according to certain embodiments.

FIG. 8 depicts a block diagram of a computing system according to certain embodiments.

### DETAILED DESCRIPTION

Methods for estimating a minimum inhibitory concentration of an antibiotic for a bacterial species are provided. In embodiments, methods include obtaining cytometric data for a plurality of test samples and a control sample for the antibiotic and bacterial species, computing distance values that reflect a measure of variation between one or more pairs of samples, and assigning a minimum inhibitory concentration based on the computed distance values. In some instances, methods include fitting a curve to a plot of the distance values of the plurality of samples versus corresponding antibiotic concentrations of the plurality of samples, and assigning a minimum inhibitory concentration based on the fitted curve. In other instances, methods include assigning cells of each sample to clusters of cell populations based on cytometric data from cells in each sample, matching clusters of cell populations from each sample with corresponding clusters of cell populations from one or more other samples, computing distance values between corresponding clusters of cell populations from pairs of samples based on cytometric data from cells in each cluster, and computing distance values between samples based on distance values between corresponding clusters of each sample. Where desired, methods also include determining a susceptibility or resistance (i.e., susceptibility, intermediate, resistance or SIR) of the antibiotic for the bacterial species based on a minimum inhibitory concentration. Systems and computer-readable media for estimating a minimum inhibitory concentration of an antibiotic for a bacterial species are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods for Estimating a Minimum Inhibitory Concentration of an Antibiotic for a Bacterial Species

As reviewed above, methods for estimating a minimum inhibitory concentration of an antibiotic for a bacterial species are provided. In particular, the present disclosure includes methods of estimating a minimum inhibitory concentration of an antibiotic for a bacterial species comprising obtaining cytometric data for a plurality of test samples and a control sample for the antibiotic and bacterial species, computing distance values that reflect a measure of variation between one or more pairs of samples, and assigning a minimum inhibitory concentration based on the computed distance values. By “minimum inhibitory concentration”, it is meant the lowest concentration of an antibiotic that inhibits observable growth of bacteria cells belonging to a bacterial species.

In some instances, methods include fitting a curve to a plot of the distance values of the plurality of samples versus corresponding antibiotic concentrations of the plurality of samples, and assigning a minimum inhibitory concentration based on the fitted curve. In other instances, methods include assigning cells of each sample to clusters of cell populations based on cytometric data from cells in each sample, matching clusters of cell populations from each sample with corresponding clusters of cell populations from one or more other samples, computing distance values between corresponding clusters of cell populations from pairs of samples based on cytometric data from cells in each cluster, and computing distance values between samples based on distance values between corresponding clusters of each sample.

Estimating a minimum inhibitory concentration of an antibiotic with respect to a bacterial species according to the subject methods results in an objective method of automatically quantifying the effect of an antibiotic on a bacterial species based on cytometric data. In particular, the subject methods do not rely on subjective decisions regarding cytometric data, such as, for example, subjective decisions regarding gating populations of the cytometric data for comparison or subjective decisions regarding choosing conditions that appear different in scatter plots of the cytometric data. As such, the subject methods facilitate making reproduceable estimates of a minimum inhibitory concentration of an antibiotic with respect to a bacterial species. Embodiments of the invention also facilitate making reproduceable estimates of a minimum inhibitory concentration as well as the susceptibility or resistance (i.e., SIR) of an antibiotic with respect to a bacterial species.

### Bacterial Test Samples

In embodiments, a test sample is a bacterial sample, by which it is meant that the test sample includes a bacteria, the susceptibility of which to a given antibiotic is to be tested. Test samples may be obtained from a variety of sources. In some instances, test samples comprise a biological sample. The term “biological sample” is used in its conventional sense to refer to a whole organism, plant, fungi or a subset of animal tissues, cells or component parts which may in certain instances be found in blood, mucus, lymphatic fluid, synovial fluid, cerebrospinal fluid, saliva, bronchoalveolar lavage, amniotic fluid, amniotic cord blood, urine, vaginal fluid and semen. As such, a “biological sample” refers to both the native organism or a subset of its tissues as well as to a homogenate, lysate or extract prepared from the organism or a subset of its tissues, including but not limited to, for example, plasma, serum, spinal fluid, lymph fluid, sections of the skin, respiratory, gastrointestinal, cardiovascular, and genitourinary tracts, tears, saliva, milk, blood cells, tumors, organs. Biological samples may be any type of organismic tissue, including both healthy and diseased tissue (e.g., cancerous, malignant, necrotic, etc.). In certain embodiments, a biological sample is a liquid sample, such as blood or derivative thereof, e.g., plasma, tears, urine, semen, etc., where in some instances the sample is a blood sample, including whole blood, such as blood obtained from venipuncture or fingerstick (where the blood may or may not be combined with any reagents prior to assay, such as preservatives, anticoagulants, etc.).

In certain embodiments the source of a sample is a “mammal” or “mammalian”, where these terms are used broadly to describe organisms that are within the class Mammalia, including the orders carnivore (e.g., dogs and cats), Rodentia (e.g., mice, guinea pigs, and rats), and primates (e.g., humans, chimpanzees, and monkeys). In some instances, the subjects are humans. The methods may be applied to samples obtained from human subjects of both genders and at any stage of development (i.e., neonates, infant, juvenile, adolescent, adult), where in certain embodiments the human subject is a juvenile, adolescent or adult. While the present invention may be applied to samples from a human subject, it is to be understood that the methods may also be carried-out on samples from other animal subjects (that is, in “non-human subjects”) such as, but not limited to, birds, mice, rats, dogs, cats, livestock and horses.

As referenced above, by “test samples” it is a meant a plurality of samples comprising, for example, bacteria belonging to a bacterial species, the susceptibility of which to a given antibiotic is to be tested. Any convenient number of bacterial cells may be included in each test sample, such as one or more bacterial cells, such as 1,000 bacterial cells or more, such as 100,000 bacterial cells or more, such as 1,000,000 bacterial cells or more, such as 100,000,000 bacterial cells or more. In other cases, any convenient range of bacterial cells may be included in each test sample, such as between 1 and 1,000 bacterial cells, such as between 100,000 and 200,000 bacterial cells, such as between 1,000,000 and 2,000,000 bacterial cells, such as between 100,000,000 and 200,000,000 bacterial cells. In some cases, the bacterial cells in test samples are measured based on the concentration of bacterial cells per unit volume, such as 1 bacterial cell per 1 mL of test sample or more, such as 1,000 bacterial cells per 1 mL of test sample or more, such as 100,000 bacterial cells per 1 mL of test sample or more, such as 1,000,000 bacterial cells per 1 mL of test sample or more, such as 100,000,000 bacterial cells per 1 mL of test sample or more. In other cases, any convenient range of bacterial cells per unit volume may be included in each test sample, such as between 1 and 1,000 bacterial cells per 1 mL, such as between 100,000 and 200,000 bacterial cells per 1 mL, such as between 1,000,000 and 2,000,000 bacterial cells per 1 mL, such as between 100,000,000 and 200,000,000 bacterial cells per 1 mL. In still other cases, the number of bacterial cells in test samples are measured based on the turbidity of the medium, such as a solution, in which the bacterial cells are held, such as, for example, the turbidity of the culture in which the bacterial cells are suspended. In such instances, the degree of turbidity of the medium or solution or culture may be quantified with reference to the McFarland standards of the turbidity of a bacterial suspension. When the number of bacterial cells in a sample is quantified based on turbidity using McFarland standards, a bacterial culture is diluted to a turbidity corresponding to any convenient McFarland standard for experimental testing, such as, for example, 0.5 McFarland, 1 McFarland, 2 McFarland, 3 McFarland or 4 McFarland. In some cases, once a bacterial culture is diluted to a turbidity corresponding to 0.5 McFarland, the bacterial culture may then be further diluted before adding staining reagents so that the final dilution corresponds to a ratio of 1 to 25 of the 0.5 McFarland suspension. In instances, the measurement of bacterial cells in test samples reflects a number or concentration or turbidity corresponding to live or viable bacterial cells.

In some embodiments, test samples have been exposed to varying concentrations of an antibiotic. In some instances, the number of test samples may comprise two or more samples, such as three or more samples, such as five or more samples, such as ten or more samples, such as 100 or more samples, such as 1,000 or more samples.

While it need not always be the case, in some cases, each sample may be substantially similar to each other test sample prior to exposing the test samples to the antibiotic, such that test samples may differ from one another substantially exclusively with regard to their exposure to an antibiotic. That is, in some cases, each test sample may derive from the same source and may comprise a substantially similar number of bacterial cells of the bacterial species, and the contents of the test samples other than the bacterial cells of the bacterial species may be substantially similar. In such cases, each test sample may be of a substantially similar volume as each other sample. In some embodiments, the plurality of samples may be treated with differing concentrations of an antibiotic, for example, in some cases, each test sample of the plurality of test samples is treated with a different concentration of an antibiotic. That is, in such cases, the test samples are configured such that the test samples comprise a gradient of differing concentrations of the antibiotic. In some cases, each step of the gradient of antibiotic concentrations in each test sample is constant, and in other cases, the steps of the gradient of antibiotic concentrations of test samples is not constant and may vary, for example the gradient of antibiotic concentrations of test samples may vary logarithmically or geometrically. In examples, the difference in antibiotic concentration between any two given test samples may vary as desired and may comprise antibiotic dilutions with a ratio of 1 to 2 between samples across a range known to be inhibitory with respect to each antibiotic-bacterial species combination. In some instances, the difference between antibiotic concentrations of test samples comprises dilutions ranges, such as, relative diluted antibiotic concentrations of 0, 4, 8, 16, 32 and 64, or, in other instances, 0, 1, 2, 4, 8, 16 and 32, or, in other instances, 0, 0.5, 1, 2 and 4, or, in other instances, 0, 0.25, 0.5, 1, 2, 4 and 8.

By a range known to be inhibitory with respect to each antibiotic-bacterial species combination, it is meant that reference may be made to a recognized standard regarding the susceptibility or resistance of the bacterial species to the antibiotic. In some cases, such recognized guidance comprises one or more values of an antibiotic concentration for a given antibiotic-bacterial species combination. That is, such guidance may comprise susceptibility, intermediate or resistance (i.e., SIR) values for an antibiotic-bacterial species combination. In some cases, guidance regarding the susceptibility or resistance of a bacterial species to an antibiotic may be provided by established organizations, such as the Clinical & Laboratory Standards Institute (CLSI) or the European Society of Clinical Microbiology and Infectious Diseases (EUCAST). In embodiments, when guidance regarding the susceptibility or resistance of the antibiotic and the bacterial species is known (e.g., when the CLSI has promulgated one or more SIR values for the antibiotic and the bacterial species), the antibiotic concentrations of the test samples may comprise concentrations that include and/or overlap with the concentrations provided in such guidance.

For example, in some cases, the plurality of test samples may comprise six different test samples such that the first test sample is exposed to an antibiotic at a concentration of 0.500 µg/mL; the second test sample is exposed to an antibiotic at a concentration of 1.000 µg/mL; the third test sample is exposed to an antibiotic at a concentration of 2.000 µg/mL; the fourth test sample is exposed to an antibiotic at a concentration of 4.000 µg/mL, the fifth test sample is exposed to an antibiotic at a concentration of 8.000 µg/mL; and the sixth test sample is exposed to an antibiotic at a concentration of 16.000 µg/mL.

In some cases, bacteria cells of test samples may belong to bacterial species of clinical significance, including, but not limited to, for example, Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Bartonella henselae, Bartonella Quintana, Bordetella bronchiseptica, Bordetella pertussis, Borrelia burgdorferi, Brucella abortus, Brucella melitensis, Brucella suis, Burkholderia, Burkholderia mallei, Burkholderia pseudomallei, Burkholderia cepacian, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Clostridium perfringens, Clostridium tetani, Corynebacterium diphtheriae, Corynebacterium fusiforme, Coxiella burnetiid, Ehrlichia chaffeensis, Ehrlichia ewingii, Eikenella corrodens, Enterobacter aerogenes, Enterobacter cloacae, Enterococcus avium, Enterococcus durans, Enterococcus faecalis, Enterococcus faecium, Enterococcus gallinarum, Enterococcus maloratus, Escherichia coli, Fusobacterium necrophorum, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Klebsiella oxytoca, Lactobacillus acidophilus, Lactobacillus bulgaricus, Lactobacillus casei, Lactococcus lactis, Legionella pneumophila, Leishmania donovani, Leptospira interrogans, Leptospira noguchii, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma Mexican, Neisseria gonorrhoeae, Neisseria meningitidis, Pasteurella multocida, Pasteurella tularensis, Peptostreptococcus, Porphyromonas gingivalis, Prevotella melaninogenica, Proteus mirabilis, Pseudomonas aeruginosa, Rhizobium radiobacter, Rickettsia prowazekii, Rickettsia psittaci, Rickettsia quintana, Rickettsia rickettsii, Rickettsia trachomae, Rochalimaea, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema pallidum, Treponema denticola, Thiobacillus, Vibrio cholerae, Vibrio comma, Vibrio parahaemolyticus, Vibrio vulnificus, Wolbachia, Yersinia enterocolitica, Yersinia pestis or Yersinia pseudotuberculosis.

By “antibiotic,” it is meant any substance that kills or inhibits the growth of bacteria. Antibiotics may be bactericidal or bacteriostatic. Antibiotics may be naturally occurring, or produced naturally, or may be synthetic. Antibiotics may be effective against one or more bacterial species; that is, antibiotics may be broad spectrum or narrow spectrum antibiotics. Though this need not always be the case, in some cases, antibiotics may refer to substances used in the practice of medicine, such as to treat or prevent bacterial infections in human patients.

Any antibiotic of interest may be applied to test samples, including known antibiotics or yet to be developed antibiotics. In some cases, antibiotics may be of clinical significance, including, but not limited to, for example, the following generic names of antibiotics: Amikacin, Gentamicin, Kanamycin, Neomycin, Netilmicin, Tobramycin, Paromomycin, Streptomycin, Spectinomycin, Geldanamycin, Herbimycin, Rifaximin, Loracarbef, Ertapenem, Doripenem, Imipenem/Cilastatin, Meropenem, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, Ceftobiprole, Teicoplanin, Vancomycin, Telavancin, Dalbavancin, Oritavancin, Clindamycin, Lincomycin, Daptomycin, Azithromycin, Clarithromycin, Erythromycin, Roxithromycin, Telithromycin, Spiramycin, Fidaxomicin, Aztreonam, Furazolidone, Nitrofurantoin, Linezolid, Posizolid, Radezolid, Torezolid, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole, Sulfonamidochrysoidine, Demeclocycline, Doxycycline, Metacycline, Minocycline, Oxytetracycline, Tetracycline, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole or Trimethoprim. In still other cases, antibiotics of interest may include: Ampicillin-Clavulanate, Ceftriaxone, Ceftazidime-Avibactam, Meropenem-Vaborbactam or Bactrim.

The subject methods may be applied to any bacterial species and antibiotic combination of interest, including, but not limited to, for example, methicillin-resistant Staphylococcus aureus and Vancomycin or methicillin-resistant Staphylococcus aureus and Teicoplanin or methicillin-resistant Staphylococcus aureus and Linezolid or methicillin-resistant Staphylococcus aureus and Daptomycin or methicillin-resistant Staphylococcus aureus and Trimethoprim/sulfamethoxazole or methicillin-resistant Staphylococcus aureus and Doxycycline or methicillin-resistant Staphylococcus aureus and Ceftobiprole or methicillin-resistant Staphylococcus aureus and Ceftaroline or methicillin-resistant Staphylococcus aureus and Clindamycin or methicillin-resistant Staphylococcus aureus and Dalbavancin or methicillin-resistant Staphylococcus aureus and Fusidic acid or methicillin-resistant Staphylococcus aureus and Mupirocin or methicillin-resistant Staphylococcus aureus and Omadacycline or methicillin-resistant Staphylococcus aureus and Oritavancin or methicillin-resistant Staphylococcus aureus and Tedizolid or methicillin-resistant Staphylococcus aureus and Telavancin or methicillin-resistant Staphylococcus aureus and Tigecycline or Pseudomonas aeruginosa and Aminoglycosides or Pseudomonas aeruginosa and Carbapenems or Pseudomonas aeruginosa and Ceftazidime or Pseudomonas aeruginosa and Cefepime or Pseudomonas aeruginosa and Ceftobiprole or Pseudomonas aeruginosa and Ceftolozane/tazobactam or Pseudomonas aeruginosa and Piperacillin/tazobactam or Pseudomonas aeruginosa and Ticarcillin/clavulanic acid or vancomycin-resistant Enterococcus and Linezolid or vancomycin-resistant Enterococcus and Streptogramins or vancomycin-resistant Enterococcus and Tigecycline or vancomycin-resistant Enterococcus and Daptomycin or any combination of the bacterial species set forth herein and the antibiotics set forth herein or any combination of bacterial species and antibiotic not as yet studied.

By “control sample” it is meant a sample of bacterial cells of the bacterial species that is not exposed to the antibiotic. In instances, a control sample may comprise more than one samples of bacterial cells of the bacterial species that is not exposed to the antibiotic, for example, replicate control samples and/or stained and unstained control samples. While it need not always be the case, in some cases, the control sample may be substantially similar to each test sample prior to exposing the test samples to the antibiotic. That is, in some cases, the control sample may comprise substantially similar number of bacterial cells of the bacterial species as the test samples, and the contents of the test sample other than the bacterial cells of the bacterial species may be substantially similar to that of the test samples. In some cases, the control sample may be of a substantially similar volume as each test sample. In other words, in some cases, the control sample may be substantially identical to the test samples in all respects, including with respect to the methods of preparation thereof, except for the application of the antibiotic to the control sample.

Embodiments of the subject method may comprise preparing the plurality of test samples and the control sample. Any convenient manner of preparing the test samples and the control sample may be employed. For example, the plurality of test samples and the control sample may be prepared such that they conform to descriptions of test samples and the control sample described above. In some cases, preparing the plurality of test samples and the control sample comprises treating a plurality of samples comprising bacterial cells of the bacterial species with the antibiotic at a plurality of different concentrations of the antibiotic and the control sample is not treated with the antibiotic. By “treating” bacterial cells of the test samples to the antibiotic, it is meant exposing the bacterial cells of the test samples to the antibiotic, for example, exposing bacterial cells to the antibiotic in a controlled manner.

A person skilled in the art would appreciate that the test samples and control sample of the subject methods may be prepared in ways and/or may consist of properties other than those described herein and that the present disclosure does not depend on a specific technique for preparing, or specific characteristics of, the test samples and control sample.

### Cytometric Data

In some embodiments, the cytometric data in the instant method may be flow cytometer data having parameters of particles (i.e., particles of the test samples and control samples, such as, for example, particles that are bacterial cells) in a sample generated from detected light. By “flow cytometer data” it is meant information regarding parameters of the particles in a flow cell that is collected by any number of detectors in a flow cytometer. In embodiments, flow cytometer data may be received from a forward scatter detector. A forward scatter detector may, in some instances, yield information regarding the overall size of a particle. In embodiments, the flow cytometer data may be received from a side scatter detector. A side scatter detector may, in some instances, be configured to detect refracted and reflected light from the surfaces and internal structures of the particle, which tends to increase with increasing particle complexity of structure. In embodiments, the flow cytometer data may be received from a fluorescent light detector. A fluorescent light detector may, in some instances, be configured to detect fluorescence emissions from fluorescent molecules, e.g., labeled specific binding members (such as labeled antibodies that specifically bind to markers of interest) associated with the particle in the flow cell. The flow cytometer data may comprise data received from one or more of a forward scatter detector, a side scatter detector as well as a fluorescent light detector. For example, in certain instances, the flow cytometer data may exclusively comprise data received from a forward scatter detector and a side scatter detector. In other instances, for example, the flow cytometer data may comprise data detected from a forward scatter detector, a side scatter detector and a fluorescent light detector.

Markers of interest may be any analyte, including analytes of biological and/or non-biological origin (e.g., chemical and/or synthetic analytes). Examples of analytes of interest include, but are not limited to, peptides, polypeptides, proteins, such as a fusion protein, a modified protein, such as a phosphorylated, glycosylated, ubiquitinated, SUMOylated, or acetylated protein, or an antibody, polysaccharides, nucleic acids, such as an RNA, DNA, PNA, CNA, HNA, LNA or ANA molecule, aggregated biomolecules, small molecules, vitamins, drug molecules, chemicals, heavy metals, pathogens and combinations thereof. In certain aspects, markers of interest may generally refer to an organic biomolecule that is differentially present in a sample of one phenotypic status (e.g., a bacterial cell affected by an antibiotic) as compared with another phenotypic status (e.g., a bacterial cell unaffected by an antibiotic).

Any convenient fluorescent molecule may be employed in the subject methods, including any substance which can absorb energy of an appropriate wavelength and emit or transfer energy. Fluorescent molecules of interest may include fluorescent dyes, semiconductor nanocrystals, lanthanide chelates, and green fluorescent protein. Fluorescent dyes may include, but are not limited to, fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2, Cy3, Cy3.5, Cy5, Cy5.5, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Alexa Fluor 700, 7-amino-4-methylcoumarin-3-acetic acid, BODIPY FL, BODIPY FL-Br.sub.2, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, conjugates thereof, and combinations thereof. Lanthanide chelates of interest include, but are not limited to, europium chelates, terbium chelates and samarium chelates. The term “green fluorescent protein” refers to both native Aequorea green fluorescent protein and mutated versions that have been identified as exhibiting altered fluorescence characteristics.

In embodiments in which the flow cytometer data comprises data detected from a fluorescent light detector, any convenient marker and/or fluorescent molecule or dye may be used for a given antibiotic-bacterial species pair. In some cases, the selection of fluorescent dye for use with an antibiotic-bacterial species pair may be based on whether the bacterial species is Gram-positive or Gram-negative. That is, whether the bacterial species gives a positive result or a negative result when bacteria belonging to the bacterial species are subjected to a Gram stain test. In some embodiments, the choice of fluorescent molecule or dye employed in the subject methods may depend only on the Gram positive or Gram negative status of the bacterial species. In such cases, the specific antibiotic in the given antibiotic-bacterial species pair would not affect the selection of fluorescent molecule or dye used for the given antibiotic-bacterial species pair. For example, in instances where the bacterial species are Gram positive, a DiOC dye may be selected, and in instances where the bacteria are Gram negative, a DiBAC dye may be selected.

In certain embodiments, methods include detecting fluorescence from a sample with one or more fluorescence detectors, such as two or more, such as three or more, such as four or more, such as five or more, such as six or more, such as seven or more, such as eight or more, such as nine or more, such as ten or more, such as 15 or more and including 25 or more fluorescence detectors. In embodiments, each of the fluorescence detectors is configured to generate a fluorescence data signal. Fluorescence from the sample may be detected by each fluorescence detector, independently, over one or more of the wavelength ranges of 200 nm -1200 nm. In some instances, methods include detecting fluorescence from the sample over a range of wavelengths, such as from 200 nm to 1200 nm, such as from 300 nm to 1100 nm, such as from 400 nm to 1000 nm, such as from 500 nm to 900 nm and including from 600 nm to 800 nm. In other instances, methods include detecting fluorescence with each fluorescence detector at one or more specific wavelengths. For example, the fluorescence may be detected at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof, depending on the number of different fluorescence detectors in the subject light detection system. In certain embodiments, methods include detecting wavelengths of light which correspond to the fluorescence peak wavelength of certain fluorophores present in a sample. In embodiments, flow cytometer data is received from one or more light detectors (e.g., one or more detection channels), such as two or more, such as three or more, such as four or more, such as five or more, such as six or more and including eight or more light detectors (e.g., eight or more detection channels).

In practicing methods according to certain embodiments, cytometric data may comprise data having been obtained from: a sample having particles (i.e., particles of the test samples and control samples, such as, for example, particles that are bacterial cells) irradiated with a light source such that light from the sample may be detected to generate measures of variation between samples based at least in part on the measurements of the detected light. As described in greater detail below, computing distance values between one or more pairs of samples refers to computing measures of variation between such one or more pairs of samples based on such light detected from samples. Various computed distance values or distance metrics may be used in practicing the subject methods.

In practicing the subject methods, cytometric data may comprise data having been obtained from: a sample having particles (e.g., in a flow stream of a flow cytometer) irradiated with light from a light source. In some embodiments, the light source is a broadband light source, emitting light having a broad range of wavelengths, such as for example, spanning 50 nm or more, such as 100 nm or more, such as 150 nm or more, such as 200 nm or more, such as 250 nm or more, such as 300 nm or more, such as 350 nm or more, such as 400 nm or more and including spanning 500 nm or more. For example, one suitable broadband light source emits light having wavelengths from 200 nm to 1500 nm. Another example of a suitable broadband light source includes a light source that emits light having wavelengths from 400 nm to 1000 nm. Where methods include irradiating with a broadband light source, broadband light source protocols of interest may include, but are not limited to, a halogen lamp, deuterium arc lamp, xenon arc lamp, stabilized fiber-coupled broadband light source, a broadband LED with continuous spectrum, superluminescent emitting diode, semiconductor light emitting diode, wide spectrum LED white light source, an multi-LED integrated white light source, among other broadband light sources or any combination thereof.

In other embodiments, cytometric data may comprise data having been obtained from: irradiating a sample with a narrow band light source emitting a particular wavelength or a narrow range of wavelengths, such as for example with a light source which emits light in a narrow range of wavelengths like a range of 50 nm or less, such as 40 nm or less, such as 30 nm or less, such as 25 nm or less, such as 20 nm or less, such as 15 nm or less, such as 10 nm or less, such as 5 nm or less, such as 2 nm or less and including light sources which emit a specific wavelength of light (i.e., monochromatic light). Where cytometric data comprises data obtained from irradiating a sample with a narrow band light source, narrow band light source protocols of interest may include, but are not limited to, a narrow wavelength LED, laser diode or a broadband light source coupled to one or more optical bandpass filters, diffraction gratings, monochromators or any combination thereof.

In some instances, the cytometric data according to the subject methods is multi-parametric cytometry data. By multi-parametric cytometric data, it is meant that the cytometric data consists of measurements of more than one characteristic of observed particles in the flow stream. For example, in some cases, multi-parametric cytometric data may consist of any combination of measurements of light that is forward scattered, side scattered and emitted from one or more types of fluorescent molecules. In some embodiments, the cytometric data comprises light scatter or marker data or a combination thereof. In such embodiments, the marker data comprises fluorescent light emission data. That is, by marker data, it is meant light emitted from, for example, fluorescent dyes used to label particles or components thereof in the sample. In some cases, the fluorescent light emission data comprises frequency-encoded fluorescence data from cells.

In some embodiments of the subject method, obtaining cytometric data from the plurality of test samples and the control sample comprises flow cytometrically analyzing the plurality of test samples and control sample. Any convenient technique for flow cytometrically analyzing the plurality of test samples and control sample may be applied, such as techniques that include any aspect of flow cytometric analysis described herein.

### Curve Fitting Method

In some embodiments, assigning a minimum inhibitory concentration based on the computed distance values comprises fitting a curve to a plot of the distance values of the plurality of samples versus corresponding antibiotic concentrations of the plurality of samples, and assigning a minimum inhibitory concentration based on the fitted curve. As described above, computed distance values represent a measure of variation among the test samples and control sample based on the cytometric data for the samples. That is, in some cases, a distance value is a metric indicating the degree to which two samples differ from each other such that a larger distance value indicates a greater difference between two samples. By a difference between two samples, it is meant a difference in the characteristics of the samples where such characteristics are observed and reflected in the cytometric data for the samples. For example, differing characteristics between samples may include the morphology of bacterial cells, which characteristics have been observed and measurements based on such observations are reflected in the cytometric data for the samples.

### Computing Distance Values in Curve Fitting Method

In embodiments, computed distance values may be based on probability binning. Probability binning may entail a process generally similar to generating one or more histograms. Bins or categories or ranges of cytometric data values (such as parameter values of multi-parametric data comprising cytometric data) may be determined. The ranges of data values for each bin may be based on the cytometric data for one or more samples. For example, such ranges of data values may be based on cytometric data that includes, as described above, measurements of detected light, such as measurements corresponding to one or more of forward scatter data, side scatter data or fluorescence data, or combinations thereof for the cytometric data for one or more samples. In some cases, bins may be assigned based on cytometric data corresponding to the control sample. For example, in some cases the cytometric data corresponding to the control sample may comprise a plurality of observed measurement values (i.e., forward scatter data, side scatter data or fluorescence data, as described above). The observed measurements may be referred to as data points and may correspond to particles, such as bacterial cells in the control sample. In some cases, bins may be assigned ranges of data values, such as ranges of cytometric data values, such that a substantially equal number of data points of the control sample would be classified into each bin. Any convenient number of bins may be used, and the number of bins may vary based on characteristics of the cytometric data. That is, in some cases the subject method may comprise setting ranges of cytometric data observed from cells in the control sample to a plurality of bins so that nearly equal numbers of cells in the control sample can be assigned to each bin in the plurality of bins, assigning cells in one of the test samples to the plurality of bins based on cytometric data detected from cells in the test sample, and computing a distance between the test sample and the control sample based on the cells in the test sample assigned to each bin.

Techniques for adaptively binning events (i.e., data points of the cytometric data), including assigning ranges of values to a collection of bins such that nearly or substantially equal numbers of data points of a sample (such as the control sample) are classified in each bin are described in Mario Roederer, Spectral compensation for flow cytometry: Visualization artifacts, limitations, and caveats, Journal of Quantitative Cell Science, at Vol. 45, Issue 3, pp. 194-205, the entirety of which is incorporated herein by reference.

Once a collection of bins and associated ranges of data are determined, cytometric data from each test sample may be, separately, assigned to each bin. As such, a test sample may be characterized based on how the data points of the test sample (i.e., the observed measurements contained in the cytometric data corresponding to such test sample) are distributed among the collection of bins. That is, while the data points of the cytometric data corresponding to the control sample may be distributed nearly equally among the bins, the data points of the cytometric data corresponding to a test sample may or may not be so distributed. For example, in certain cases, the data points for a test sample may be skewed toward one or more bins. Such characteristics of how data points are distributed among the bins may be mathematically formalized. In particular, in embodiments, a distance value for each test sample may be computed based on how the cytometric data, i.e., data points, for each test sample are allocated among the different bins. That is, computed distance values may be based on probability binning, meaning a distance value may be computed, such that the distance value represents how the cytometric data of a test sample are assigned to bins.

In embodiments of the subject method, probability binning may be based on a chi-squared statistic. That is, in some cases, the known statistical technique, a chi-squared statistic or a chi-squared test, may be applied to cytometric data corresponding to a test sample, where such chi-squared statistic characterizes the cytometric data corresponding to the test sample. For example, the chi-squared statistic for a test sample may indicate the existence of a significant difference between the distribution of data values from a test sample versus the distribution of data values from the control sample and/or may indicate a degree of significance of such difference. Alternatively, the chi-squared statistic may indicate that the cytometric data corresponding to a test sample is statistically the same as, or not meaningfully different from, the cytometric data corresponding to the control sample.

In some embodiments, computed distance values are based on a T statistic. The T statistic is a known, specially developed, distance metric, which can be applied to compute the distance between - i.e., compute a numerical representation of the degree of difference exhibited between - a test sample and the control sample, as such samples are represented in the histograms, as described above. The T statistic method is based on an adaptation of the chi-squared statistic and can be applied to cytometric data that is comprised of data from all flow channels from which data are collected, i.e., forward scatter data, side scatter data and/or fluorescence data, as described above. The T statistic is described in Keith A. Baggerly, Probability binning and testing agreement between multivariate immunofluorescence histograms: Extending the chi-squared test, Journal of Quantitative Cell Science, at Vol. 45, Issue 2, pp. 141-50, the entirety of which is incorporated herein by reference.

### Plotting Distance Values in Curve Fitting Method

In embodiments, a plot of distance values of the plurality of samples versus corresponding antibiotic concentrations of the samples may consist of a collection of data points on a two-dimensional plot. Each data point may correspond to a sample and may comprise (i) a distance value reflecting the computed distance between a sample and the control sample and (ii) the antibiotic concentration of such sample. In such embodiments, the plot is a two dimensional plot with one axis, such as the y-axis, representing distance values of samples and another axis, such as the x-axis, representing concentrations of the antibiotic applied to samples.

Fitting a curve to such plot comprises deriving a curve, such as deriving a curve represented by a mathematical function, that approximates the relationship among the data points of the plot. Such curve may be used to estimate certain distance values. That is, such curve may be used to estimate distance values corresponding to antibiotic concentrations where there are no data points - i.e., no corresponding test sample exposed to such antibiotic concentration. That is, when the cytometric data does not include a distance value for a sample at a particular antibiotic concentration, the distance value at such antibiotic concentration may be inferred based on the fitted curve. Any convenient mathematical function and/or curve fitting process or algorithm may be applied. For example, in some embodiments, the fitted curve may comprise a polynomial function, including a first degree polynomial, a second degree polynomial, or a third degree polynomial or a polynomial of a degree higher than three. In other embodiments, the fitted curve may comprise a trigonometric function or a sigmoid function or another function. The fit of the curve may be measured — and determined or optimized — in any convenient way, such as ordinary least squares or total least squares or some other measurement of fit between the fitted curve and the plotted data points.

In some embodiments, the curve fitted to the plot of the distance values of the plurality of samples versus corresponding antibiotic concentrations of the plurality of samples is a logistic curve. That is, the fitted curve can be described by a logistic function, such that the curve appears substantially “S” shaped and may also be referred to as a sigmoid curve. The “S” shaped logistic curve comprises both a lower horizontal asymptote of the curve as x values approach negative infinity and an upper horizontal asymptote of the curve as x values approach positive infinity. The logistic curve may be described by the following mathematical formula:

$$f \left(x\right) = \frac{L}{\left(1 + {e}^{- k \left(x - {x}_{0}\right)}\right)}$$

In such formula, L is the curve’s maximum value; x₀ is the x value corresponding to the sigmoid’s midpoint; and k is the logistic growth rate of the curve. In some cases, fitting a logistic curve to the plot of the distance values of the plurality of samples comprises arriving at appropriate values for L, x₀ and k in the above formula according to any desirable curve fitting process, such as those described above. In instances, the lower horizontal asymptote of the fitted logistic curve is assigned a distance of zero. That is, the y-value of the curve corresponding to the horizontal asymptote of the curve as x values approach negative infinity is set to zero, meaning the distance value of such bacterial cells is zero or that such cells are not distinguishable from the control sample. In some cases, the upper horizontal asymptote of the fitted logistic curve represents concentrations of the antibiotic at which substantially the entire sample is affected by the antibiotic. By a sample being affected by the antibiotic, it is meant that the antibiotic has the effect of killing or inhibiting the growth of substantially all of the bacterial cells in the sample. As such, a distance value sufficiently near the value of the upper horizontal asymptote of the fitted logistic curve indicates that substantially all of the bacterial cells of a sample at the corresponding concentration of the antibiotic would be killed or their growth would be inhibited by the antibiotic.

### Assigning a MIC in Curve-Fitting Method

Subject methods further comprise assigning a minimum inhibitory concentration (MIC) based on the fitted curve. By this, it is meant that the concentration corresponding to the minimum inhibitory concentration of the antibiotic with respect to the bacterial species is assigned based on one or more characteristics of the fitted curve. In some cases, the fitted curve may exhibit a particular shape or a particular mathematical property or some other distinguishing feature corresponding to a specific concentration, and it is at the antibiotic concentration corresponding to such property of the curve that the minimum inhibitory concentration may be assigned. As an example, a first curve fitted to one combination of a bacterial species and antibiotic pair may differ from a second curve fitted to a different combination of a bacterial species and antibiotic pair. Notwithstanding the differences in the first and second fitted curves, both fitted curves curve may nonetheless exhibit the same particular shape, mathematical property or other distinguishing feature. The first fitted curve may exhibit such particular shape, mathematical property or other distinguishing feature at a first antibiotic concentration and the second fitted curve may exhibit such particular shape, mathematical property or other distinguishing feature at a second antibiotic concentration. Assigning a minimum inhibitory concentration based on a characteristic of the fitted curve enables an objective determination of a minimum inhibitory concentration that is also a reproducible metric.

In embodiments where the fitted curve is a logistic curve, a minimum inhibitory concentration may be assigned the antibiotic concentration corresponding to a point at which the slope of the logistic curve is a maximum. That is, the “S” shaped curve of a logistic curve is expected to have a single point where the slope of the curve at a point is a maximum, and the minimum inhibitory concentration of the antibiotic-bacterial species pair is assigned the concentration corresponding to such point. The slope of the curve at a point may be computed in any convenient manner including utilizing any convenient algorithm, such as an algorithm for calculating, such as symbolically calculating, the slope of a curve at a point or an algorithm for approximating the slope of a curve at a point. In other embodiments where the fitted curve is a logistic curve, a minimum inhibitory concentration may be the antibiotic concentration corresponding to a point which is halfway between the upper and lower horizontal asymptotes of the logistic curve. That is, the midpoint between the upper and lower asymptotes of the fitted curve is the distance corresponding to the y-value that is the half-way point between the y-value corresponding to the upper asymptote and the y-value corresponding to the lower asymptote. A horizontal line drawn at such midpoint distance intersects the fitted logistic curve at a single point. The minimum inhibitory concentration may be assigned the antibiotic concentration corresponding to such point. The antibiotic concentration at such point would be expected to be the antibiotic concentration at which growth of the bacterial species is inhibited 50%. Such technique for determining the minimum inhibitory concentration based on the midpoint between the upper and lower asymptotes of the fitted curve may find particular use when there are no replicates for the experimental conditions (dilutions) in the experiment, such as when there are no replicates of the test samples and/or control sample.

In still other embodiments where the fitted curve is a logistic curve, the minimum inhibitory concentration may be assigned the antibiotic concentration corresponding to a point that is a reliable detection limit of the curve. Such technique for determining the minimum inhibitory concentration based on a reliable detection limit of the curve may find particular use when at least three replicates per condition are run in the experiment, such as three replicates or four replicates or five replicates or ten replicates or twenty or more replicates. Such replicates may comprise duplicates or replications of the test samples and control sample, such that an experiment is conducted with multiple test samples corresponding to each antibiotic concentration as well as multiple control samples that have not been exposed to antibiotic. When at least three replicates are run in the experiment, the reliable detection limit (RDL) approximately corresponds to the antibiotic concentration at which the assay is 97.5% specific and 97.5% sensitive in the detection of growth inhibition of the bacterial cells. This is the lowest concentration where the lower limit of the 95% confidence band of the fitted curve is higher than the upper limit of the 95% confidence band at the lower asymptote of the fitted curve.

Each technique for assigning a minimum inhibitory concentration described above is based on objective analyses of the cytometric data, meaning subjective determinations or judgments are not required, and therefore offers a reproducible technique for assigning a minimum inhibitory concentration for combinations of antibiotics and bacterial species.

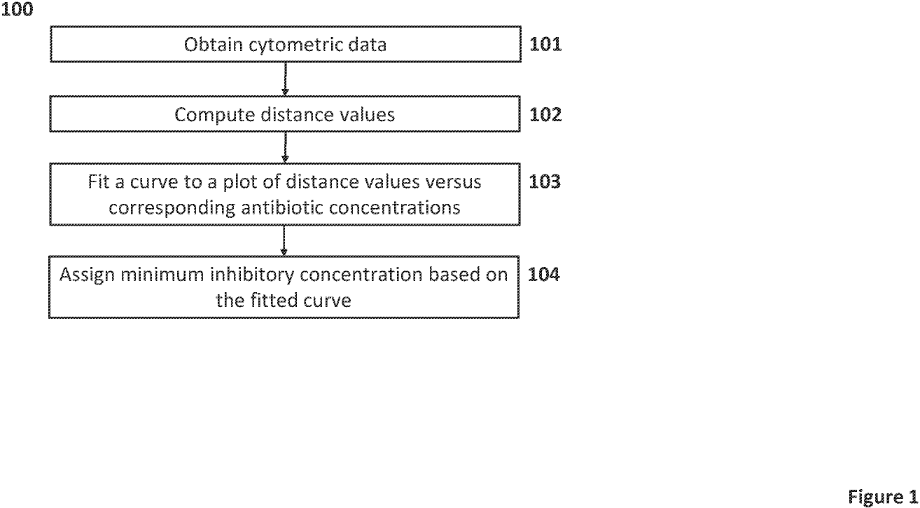

FIG. 1 presents a flowchart 100 that schematically demonstrates one exemplary instance of the subject method for determining a minimum inhibitory concentration based on cytometric data and utilizing curve-fitting as described above. The first step 101 is to obtain cytometric data for a plurality of test samples and a control sample for the antibiotic and bacterial species. The second step 102 is to compute distance values that reflect a measure of variation between one or more pairs of samples. In some cases, distance values are computed among each combinations of test samples and the control sample. The third step 103 is to fit a curve to a plot of the distance values of the plurality of samples versus corresponding antibiotic concentrations of the plurality of samples. The final step 104 is to assign a minimum inhibitory concentration based on the fitted curve.

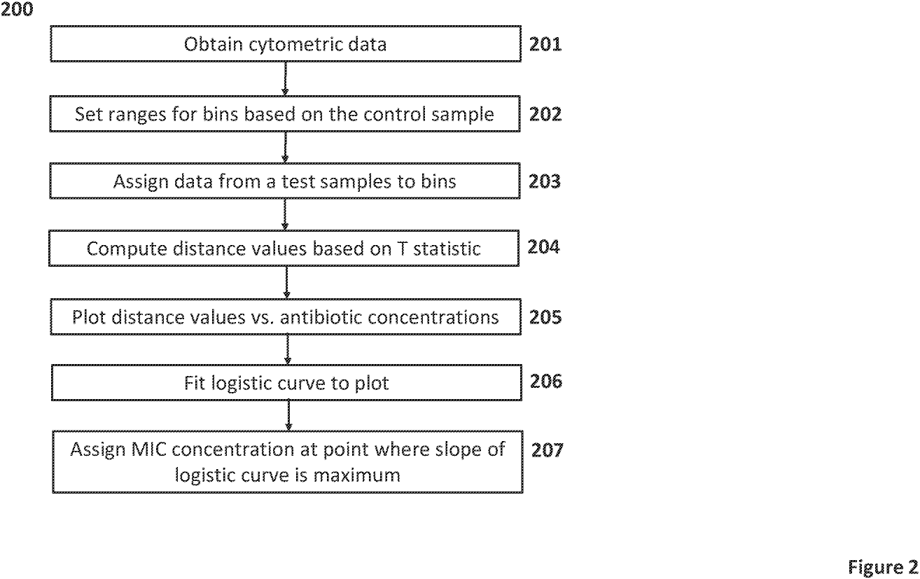

FIG. 2 presents a flowchart 200 that schematically demonstrates another exemplary instance of the subject method for estimating a minimum inhibitory concentration and utilizing curve-fitting as described above. The first step 201 is to obtain cytometric data for a plurality of test samples and a control sample for the antibiotic and bacterial species. The second step 202 is to assign ranges of values of measurement data to a plurality of bins based on the cytometric data for the control sample. The ranges of data values assigned to the bins are set such that each of the bins includes a nearly equal number of data points (e.g., the one or more parameter values measured for a bacterial cell) from the control sample are assigned to each bin in the plurality of bins. The third step 203 is, for each test sample, to assign data points from the cytometric data corresponding to the test sample to the plurality of bins. The fourth step 204 is, for each test sample, to compute a distance value using the T statistic based on at least in part on how the data points of the cytometric data from the test sample are distributed into the plurality of bins. A value of the T statistic is the computed distance between a test sample and the control sample and in some cases reflects a degree to which the cytometric data indicates that the morphology of bacterial cells in the test sample differ from the morphology of the bacterial cells in the control sample. Such differences in morphology may, in embodiments, be related to the degree to which an antibiotic is effective at killing or inhibiting the growth of bacterial cells in test samples. The fifth step 205 is to plot points on a two dimensional plot based on the computed distance values and antibiotic concentrations for each test sample where the plot is a two-dimensional plot with an x-axis corresponding to different concentrations of the antibiotic applied to each test sample and a y-axis corresponding to different computed distance values for each test sample. The sixth step 206 is to fit a logistic curve to the plot generated in step 205. The final step 207 is to assign a minimum inhibitory concentration for the pair of the bacterial species and antibiotic combination based on the logistic curve fitted to the plot in step 206. In particular, the point at which the slope of the logistic curve is a maximum is computed and the estimate of the minimum inhibitory concentration is assigned the concentration at such point.

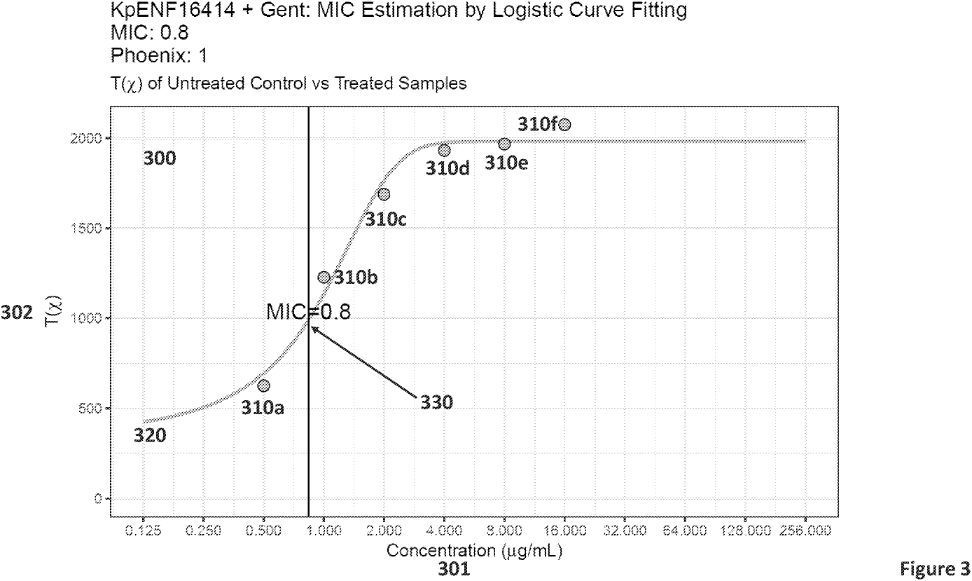

FIG. 3 shows an example of assigning a minimum inhibitory concentration for a bacterial species and antibiotic pair based on a fitted logistic curve according to embodiments of the subject method. As shown in FIG. 3, the two-dimensional plot 300 comprises an x-axis 301 representing different antibiotic concentrations and a y-axis 302 corresponding to different distance values for the test samples computed based on the T statistic. Plotted on plot 300 are points 310a-310f. Each point 310a-310f corresponds to a test sample, and each point 310a-310f is plotted such that its position on the x-axis 301 represents the antibiotic concentration of the test sample and its position on the y-axis 302 represents the distance value based on the T statistic for each test sample. The data points 310a-310f shown in FIG. 3 correspond to, for example, the result of plotting data points in step 205 of FIG. 2. Curve 320 is a logistic curve that has been fitted to the plotted data points 310a-310f. Point 330 on logistic curve 320 is the point at which the logistic curve achieves a maximum slope at a point. Since the logistic curve achieves a maximum slope at point 330, the estimated minimum inhibitory concentration is assigned to be the antibiotic concentration at point 330. The x-axis value of point 330 corresponds to an antibiotic concentration of 0.8 µg/mL. Accordingly, the estimated minimum inhibitory concentration of the antibiotic for the bacterial species of the test samples is assigned the value of 0.8 µg/mL. The determination of the estimated minimum inhibitory concentration as illustrated in plot 300 is accomplished based on objective determinations regarding the cytometric data.

### Group-Based Method

In some embodiments, estimating a minimum inhibitory concentration is based on computing distance values by assigning cells of each sample to clusters of cell populations based on cytometric data from cells in each sample, matching clusters of cell populations from each sample with corresponding clusters of cell populations from one or more other samples, computing distance values between corresponding clusters of cell populations from pairs of samples based on cytometric data from cells in each cluster and computing distance values between samples based on distance values between corresponding clusters of each sample. As described above, computed distance values represent a measure of variation among the test samples and control sample based on the cytometric data for the samples. That is, in some cases, a distance value is a metric indicating the degree to which two samples differ from each other such that a larger distance value indicates a greater difference between two samples. By a difference between two samples, it is meant a difference in the characteristics of the samples where such characteristics are reflected in the cytometric data for the samples. For example, differing characteristics between samples may include the morphology of bacterial cells, which characteristics have been observed and measurements based on such observations are reflected in the cytometric data for the samples.

### Clusters in the Group-Based Method

In embodiments, computing distance values between samples comprises assigning cells of each sample to clusters of cell populations based on cytometric data from cells in each sample. By “clustering,” it is meant that particles (e.g., bacterial cells) of a sample possess properties (for example, optical, impedance, or temporal properties) with respect to one or more measured parameters such that the measured parameter data form a cluster in the data space. In embodiments, cytometric data is comprised of signals from any given number of different parameters, such as, for instance two or more, three or more, four or more, five or more, six or more, seven or more, eight or more, nine or more, ten or more, and including 20 or more. Thus, populations are recognized as clusters in the data. Conversely, each data cluster may be interpreted as corresponding to a population of a particular type of, or morphology of, particle or cell, although clusters that correspond to noise or background typically also are observed. A cluster may be defined in a subset of the dimensions, e.g., with respect to a subset of the measured parameters, which corresponds to populations that differ in only a subset of the measured parameters or features extracted from the measurements of the particle (e.g., bacterial cell). In some cases, clusters of cell populations share similar characteristics of the parameter values of the underlying cytometric data (e.g., parameter values representing measurements of forward scattered light, side scattered light or fluorescent light) collected for such cells of the test sample. Any convenient number of clusters may be defined, and any technique or algorithm may be employed to assign cells to different clusters.

In some embodiments, assigning cells of each sample to clusters of cell populations comprises applying k-means clustering to a test sample. By “k-means clustering” it is meant the known partitioning technique that aims to partition data points for each event or cell of a test sample into k clusters so that each data point belongs to the cluster with the nearest mean. The technique of k-means clustering, including various popular embodiments that utilize k-means clustering, is further described in Lukas M. Weber and Mark D. Robinson, Comparison of Clustering Methods for High-Dimensional Single-Cell Flow and Mass Cytometry Data, Cytometry, Part A, Journal of Quantitative Cell Science, at Vol. 89, Issue 12, pp. 1084-96, the entirety of which is incorporated herein by reference. By “mean of a cluster,” it is meant a cluster center or cluster centroid, for example, in some cases, the point that represents mean values of each of the parameters comprising data points in the cluster. Variations on k-means clustering may also be employed including, but not limited to, for example, k-medians clustering or k-medoids clustering. In other embodiments, assigning cells of each sample to clusters of cell populations comprises applying a Self-Organizing Map. By “Self-Organizing Map,” it is meant applying a type of artificial neural network algorithm that, as a result of the neural network training step, produces a map, in this case, where the map comprises a collection of clusters defining the data points or cells of a sample. The technique of applying a Self-Organizing Map, including the popular embodiment of the Self-Organizing Map, FlowSOM, is further described in Lukas M. Weber and Mark D. Robinson, Comparison of Clustering Methods for High-Dimensional Single-Cell Flow and Mass Cytometry Data, Cytometry, Part A, Journal of Quantitative Cell Science, at Vol. 89, Issue 12, pp. 1084-96, the entirety of which is incorporated herein by reference.

### Matching Clusters in the Group-Based Method

As described above, in embodiments, the subject method further comprises matching clusters of cell populations from each sample with corresponding clusters of cell populations from one or more other samples. That is, once data points from test samples and the control sample are partitioned into clusters, clusters from one sample may be expected to have analogous counterpart clusters in other samples. Analogous counterpart clusters may be identified based on properties exhibited by each cluster, such as the mean, median, variance or other mathematical properties or combinations thereof for the clusters. By “matching” clusters from each sample, it is meant for a cluster in a sample, identifying a counterpart cluster from each other sample. Any convenient means of searching for, evaluating fit of, and identifying matching clusters may be employed. In some embodiments, matching corresponding clusters of cell populations from each sample comprises applying a mixed edge cover algorithm. The mixed edge cover algorithm as well as embodiments of techniques that utilize the mixed edge cover algorithm are further described in Ariful Azad, Bartek Rajwa and Alex Pothen, Immunophenotype Discovery, Hierarchical Organization, and Template-Based Classification of Flow Cytometry Samples, Frontiers in Oncology, at Vol. 6, Art. 188, pp. 1-20, the entirety of which is incorporated herein by reference. Variations on mixed edge cover algorithms may also be employed.

### Computing Distance Values in the Group-Based Method

In embodiments, the subject method further comprises computing distance values between corresponding clusters of cell populations from pairs of samples based on cytometric data from cells in each cluster. That is, once a cluster from one test sample is matched with its counterpart corresponding cluster from another test sample, the distance between this pair of clusters may be computed. In embodiments, computing distances between corresponding clusters comprises measuring a distance between a cluster from a first test sample and a corresponding cluster from other test samples and the control sample. In other words, in embodiments, distances are measured between clusters of different samples.

The computed distance value between two clusters represents an aggregated measure of the variation between the observed parameters (e.g., forward scattered light, side scattered light or fluorescent light) of the events that make up the two clusters. In some cases, the computed distance value represents a distance between the two clusters as measured on a plot on which both clusters are represented. That is, the computed distance value according to the subject method may, in some embodiments, be visualized as the distance between two clusters on a plot depicting both clusters. In some embodiments, computing distances between corresponding clusters is based on distribution parameters of each cluster. That is, clusters may be characterized by statistical measures of the data points comprising the cluster, such as, for example, a distribution parameter based on the mean or median of data points comprising a cluster.

Once clusters are characterized quantitatively, based on, for example, distribution parameters of clusters, distances between clusters may be computed based on such quantitative characterizations of clusters. That is, for example, the computed distance value may be the distance between the “center” value of one cluster and the “center” value of another cluster. The “center” value of a cluster may be any convenient statistical representation of the data comprising the cluster, and as described above, may, in some embodiments, be based on distribution parameters of a cluster such as mean or median values. In some embodiments, distance values between corresponding clusters are computed using Euclidean distance measurement. That is, a Euclidean distance computation is applied to the distribution parameters that describe two clusters. In other embodiments, distance values between corresponding clusters are computed using Mahalanobis distance measurement. That is, a Mahalanobis distance computation is applied to the distribution parameters that describe two clusters.

### Assigning an MIC in the Group-Based Method

Embodiments of the subject method comprise assigning a minimum inhibitory concentration (MIC) based on the computed distance values. As described in detail above, a minimum inhibitory concentration refers to a specific concentration of an antibiotic with respect to a bacterial species. Computed distance values refer to measures of the degree of similarity or difference between samples, such as the degree of similarity or difference in the morphology of bacterial cells as observed in the cytometric data for the samples.

Some embodiments of the subject method may further comprise assigning each sample to a branch of a hierarchical tree based on distance values between samples. A hierarchical tree may be a data structure configured to store antibiotic concentration and distance information for the samples and configured to be represented visually, such as on a display device. As such, a hierarchical tree offers a way to visualize the cytometric data. In particular, a hierarchical tree may show visually which test samples are relatively similar or relatively different from one another as well as a visual representation of the computed distances between test samples. By hierarchical tree data structure, it is meant an abstract data structure consisting, recursively, of a single root node and one or more child nodes, where the root note is connected by edges to the one or more child nodes. In some cases, the hierarchical tree may be a binary tree, meaning a tree data structure where each root node consists of no more than two child nodes. In embodiments, the hierarchical tree may be arranged such that each test sample is assigned only to terminal child nodes. The hierarchical tree structure may be further configured to be plotted on a two-dimensional plot. Each test sample that comprises a terminal child node of the hierarchical tree may be assigned a position on the x-axis of the two dimensional plot. Edges connecting test samples at terminal child nodes to a neighboring test sample at a terminal child node or to a neighboring collection of test samples at an intermediate root node may extend vertically along the y-axis. The order in which the samples are presented on the x-axis need not convey specific information characterizing the samples as it is instead the organization or topology of the hierarchical tree that reflects the relationship among samples; indeed, the order in which samples are presented along the x-axis can be changed without changing the information conveyed by the visual display of the hierarchical tree. In contrast, the y-axis of the two dimensional plot may represent computed distance values. In such cases, the height of a given edge on the y-axis may be configured to indicate the computed difference between, for example, a test sample connected to such edge and the neighboring test sample or the neighboring group of test samples. In other examples, the height of an edge on the y-axis may be configured to indicate the computed difference between, for example, a pair or a group of test samples (i.e., a pair or a group of non-terminal child nodes) connected by such edge and the neighboring pair or group of test samples. In such examples, average distance values of a group of two or more test samples and a neighboring group of test samples may be used for computation of the distance between such groups. In embodiments, the heights of edges on the y-axis of the hierarchical tree may be determined using agglomerative clustering, meaning heights of edges on the y-axis of the hierarchical tree, may be computed using a “bottom-up” approach such that each observation starts as a group (i.e., a group of a single observation or test sample), and, moving up the hierarchical tree, pairs of groups are merged, such that the height on the y-axis of each edge of the hierarchical tree is the distance between each group. A hierarchical tree plotted as described above on a two-dimensional plot so that the y-axis indicates computed distance values and edges between tree nodes are drawn so that they reflect computed distance values may offer a visual representation of the cytometric data that efficiently conveys relative similarities and differences between the cytometric data comprising test samples.

Some embodiments further comprise assigning samples to groups based on distances between samples. Groups refer to collections of test samples that share characteristics of the events (i.e., measurements of bacterial cells in the cytometric data) that comprise each test sample. Any convenient number of groups may be defined, and any convenient number of test samples may be assigned to each group. In instances, groups may be determined by finding the largest group (i.e., a group that contains the largest number of samples or terminal child nodes) that does not include a control sample (i.e., a sample not treated by antibiotics). In such instances, the resulting number of groups would be expected to equal one more than a minimum number of groups that include a control. Defining groups of samples based on finding the largest group that does not include a control sample may entail starting at the root node of the hierarchical tree and pruning branches at the nodes until there is a branch that does not contain a control sample. By “pruning branches starting from the root node of the hierarchical tree,” it is meant starting from the very top of the hierarchical tree and splitting tree branches into groups at different nodes progressing “down” the tree towards terminal child nodes, until a group of samples results from the pruning, where such group does not include a control sample. As such, the hierarchical tree is constructed using distance measurements from the “bottom up,” but samples are assigned to groups based on the hierarchical tree structure from the “top down.” As such, both the test samples and one or more control samples are assigned to groups, such that a group may be comprised of exclusively test samples or exclusively control samples or a combination of both test samples and control samples. Groups of test samples may be characterized by aggregated distance values, as described in detail above. That is, in some cases, a distance metric can apply to distances between two groups as well as distances between two samples. Any convenient method of aggregating the distance values of two groups and computing distances between groups may be employed.

In embodiments that comprise assigning samples to groups based on distances between samples, such embodiments may further comprise assigning a minimum inhibitory concentration to be the antibiotic concentration corresponding to the sample with the lowest antibiotic concentration in a first group of samples that is the furthest distance away from a second group of samples, wherein the second group of samples includes the untreated control sample. That is, the test samples and control sample are assigned to a plurality of groups, as described above, based on the computed distance values between the samples. The composition of the groups may be expected to include a group that comprises the control sample and, in some cases, one or more test samples, as well as one or more additional groups that comprise exclusively test samples. In instances in which more than one untreated control sample is analyzed and therefore included in the hierarchical tree, the resulting composition of the groups may include more than one group that comprises a control sample. According to embodiments of the subject method, an estimated minimum inhibitory concentration is assigned in part based on the characteristics of the groups of test samples. Specifically, a group of samples is identified that contains the control sample, the bacterial cells of which have not been treated with the antibiotic. This group may be referred to as the second group. After the group containing the control sample is identified, the group of test samples that is the furthest computed distance away from the group containing the control sample (i.e., the second group) is identified. The group that is the furthest computed distance away from the group containing the control sample may be referred to as the first group. That is, the first group is the group of test samples among the plurality of groups of test samples that is the furthest distance away from the second group. Within the first group of test samples, the test sample that was treated with the lowest concentration of antibiotic is identified. This antibiotic concentration — the lowest antibiotic concentration in the first group — is assigned to be the minimum inhibitory concentration for the bacterial species and antibiotic pair comprising the test samples and control sample. The minimum inhibitory concentration may be assigned as such because the first group, by definition, does not include a control sample. As such, the samples that comprise the first group reflect underlying characteristics measured flow cytometrically that are different from the underlying characteristics of the second group of samples, which does include a control. The differences in the underlying characteristics, and the resulting, corresponding distance measurements are the reason the samples in the first group cluster apart from the samples in the second group. The first group, which does not include a control, is expected to be susceptible to the antibiotic. The minimum inhibitory concentration, by definition, is the minimum concentration of the antibiotic that inhibits growth of the bacteria, and, as such, it may be assigned the minimum concentration in first group.

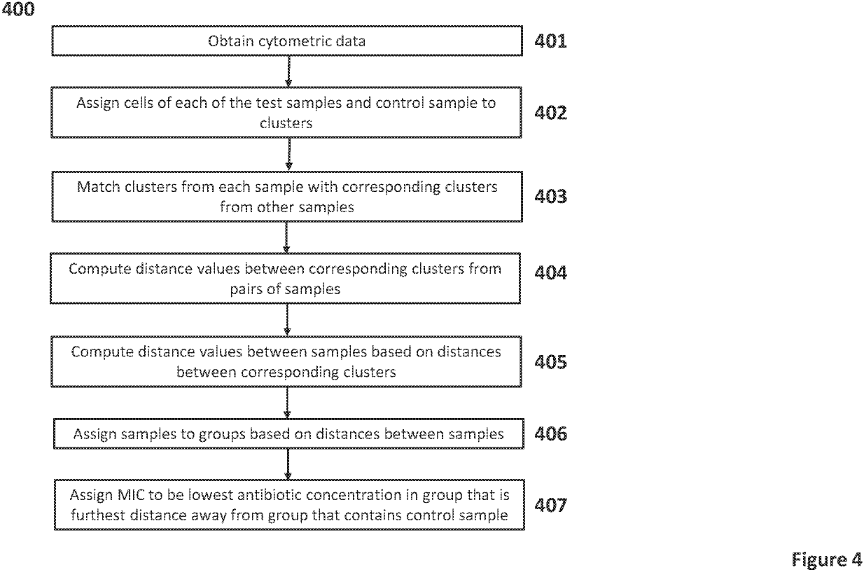

FIG. 4 presents a flowchart 400 that schematically demonstrates one exemplary instance of the subject method for determining a minimum inhibitory concentration based on cytometric data and utilizing the group-based method described above. The first step 401 is to obtain cytometric data for a plurality of test samples and a control sample for the antibiotic and bacterial species. The second step 402 is, for each sample, to assign cells to clusters of cell populations based on cytometric data from cells in each sample. As described above, any convenient technique may be used to cluster events within the test samples and control sample. The third step 403 is to match clusters of cell populations from each sample with corresponding clusters of cell populations from one or more other samples. Included in this step is identifying corresponding clusters among the samples, including matching clusters from the control sample with corresponding clusters from the test samples. The fourth step 404 is to compute distance values between corresponding clusters of cell populations from pairs of samples based on cytometric data from cells in each cluster. In embodiments, distances between corresponding clusters from each pair of samples, including pairs that include a cluster from the control sample, may be computed. The fifth step 405 is to compute distance values between samples based on distance values between corresponding clusters of each sample. That is, as described above, embodiments include computing the distance between two samples based on the distances between corresponding clusters of each sample. The sixth step 406 is to assign samples to groups based on distances between samples. As described above, in embodiments, a plurality of groups may be created, each of which may comprise one or more samples. In embodiments, the control sample is also assigned to a group. The final step 407 is to assign a minimum inhibitory concentration to be the antibiotic concentration corresponding to the sample with the lowest antibiotic concentration in a first group of samples that is the furthest distance away from a second group of samples, wherein the second group of samples includes the untreated control sample

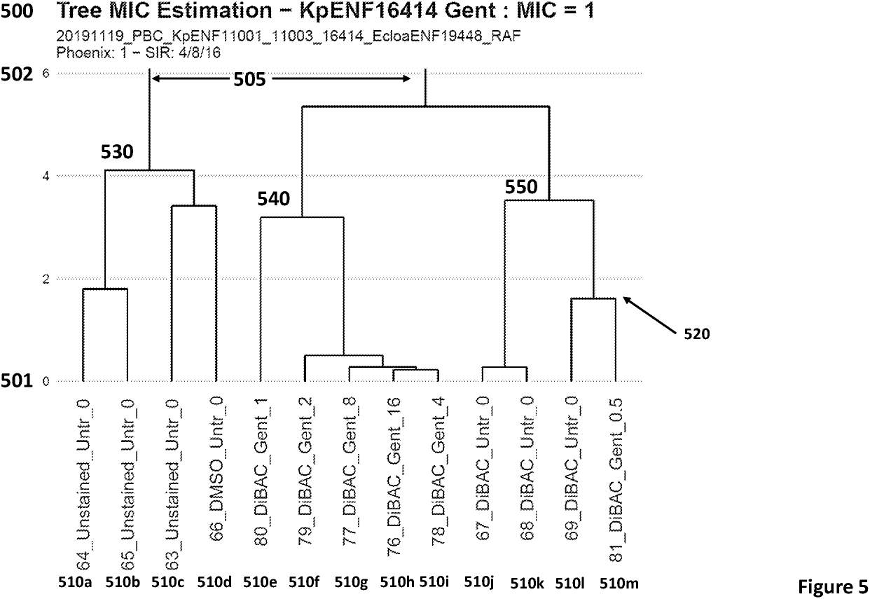

FIG. 5 shows an example of assigning a minimum inhibitory concentration for an antibiotic-bacterial species pair based on the configuration of groups of samples arranged in a hierarchical tree according to embodiments of the subject method. As shown in FIG. 5, the two-dimensional plot 500 comprises an x-axis 501 on which different test samples and the control sample are arranged and a y-axis 502 corresponding to computed distance values between samples. As described above, in embodiments, such distance values may be based on distances between corresponding clusters within the test samples and control sample. Such distances may be computed using any convenient distance metric. For example, in some cases, such distances may be computed based on Euclidean distances, and in other embodiments, such distances may be computed based on Mahalanobis distances. Plotted on plot 500 is a lower subsection of a single hierarchical tree 505 where samples are assigned to each of the terminal child nodes of the tree 510a-510m. That is, each terminal child node of the tree 510a-510m corresponds to test samples and control sample, and the y-axis 502 heights of the edges connecting samples and groups of samples are plotted to represent the computed distances between each sample and neighboring samples or groups of samples. For example, the height 520 on the y-axis 502 of the edges connecting samples 510l and 510m indicates the distance between samples 510l and 510m. The samples have been assigned to a first group 530 consisting of samples 510a-510d, a second group 540 consisting of samples 510e-510i and a third group 550 consisting of samples 510j-510m. The first group 530 and the third group 550 contain control samples, which indicates that any test samples in the first group and the second group are not sufficiently different from untreated controls to comprise a minimum inhibitory concentration. (The results of the experiment illustrated in FIG. 5 included replicate control samples, as well as unstained and stained control samples. As a result, both the first group 530 and the third group 550 contain control samples) The second group 540 does not include a control sample, and it follows, therefore, that the second group 540 is the group that is furthest distance away from a group that contains the control samples. Since sample 510e has the lowest antibiotic concentration of any of the samples contained in the second group 540, the antibiotic concentration of sample 510e is assigned to be the minimum inhibitory concentration for this antibiotic-bacterial species pair.

### Susceptibility or Resistance

In some embodiments, a susceptibility or resistance (e.g., SIR or susceptibility, intermediate, resistance categorization) of the antibiotic for the bacterial species is determined based on the minimum inhibitory concentration. The minimum inhibitory concentration of the antibiotic may be determined based on any of the techniques described herein. Such minimum inhibitory concentration corresponds to a particular, unique, pair of antibiotic and bacterial species - the antibiotic that the bacterial cells in the test samples are exposed to. Based on the estimated minimum inhibitory concentration for an antibiotic-bacterial species pair, determinations may be made about the susceptibility or resistance of the bacterial species to the antibiotic. For example, in some cases, a minimum inhibitory concentration at a concentration that is higher than expected, higher than can be clinically applied, higher than or as high as certain other antibiotic-bacterial species pairs or high based on some other measure, may indicate a resistance of a bacterial species to an antibiotic. Alternatively, in some embodiments, a minimum inhibitory concentration at a concentration that is lower than expected, low enough to be clinically applied, as low as or lower than certain other antibiotic-bacterial species pairs or high based on some other measure, may indicate a susceptibility of a bacterial species to an antibiotic. In some embodiments, a susceptibility or resistance of the antibiotic for the bacterial species can be determined by comparing the minimum inhibitory concentration determined based on the subject methods to known breakpoints. By breakpoint, it is meant a threshold concentration of an antibiotic that defines whether a bacterial species is susceptible or resistant to the antibiotic. Such known breakpoints may comprise commonly used clinical breakpoints, such as those promulgated by the Clinical and Laboratory Standards Institute (CLSI) or the European Committee for Antimicrobial Susceptibility Testing (EUCAST).

### Systems for Estimating a Minimum Inhibitory Concentration of an Antibiotic for a Bacterial Species

Aspects of the present disclosure include systems for estimating a minimum inhibitory concentration of an antibiotic for a bacterial species according to the subject methods. In some embodiments, systems include an apparatus configured to obtain cytometric data, and a processor configured to assign a minimum inhibitory concentration based on such data.

### Flow Cytometers

In certain embodiments, the apparatus is configured to obtain the cytometric data by analyzing the plurality of test samples and the control sample for the antibiotic and bacterial species. In some embodiments, the apparatus is configured to obtain cytometric data from the plurality of test samples and the control sample by flow cytometrically analyzing the plurality of test samples and control sample. For example, in embodiments, the apparatus may be configured to obtain the cytometric data from a flow cytometer. That is, in embodiments, the apparatus may be, or may be operably connected to, a flow cytometer.

In some embodiments, the subject flow cytometers have a flow cell, and a laser configured to irradiate particles in the flow cell. In embodiments, the laser may be any convenient laser, such as a continuous wave laser. For example, the laser may be a diode laser, such as an ultraviolet diode laser, a visible diode laser and a near-infrared diode laser. In other embodiments, the laser may be a helium-neon (HeNe) laser. In some instances, the laser is a gas laser, such as a helium-neon laser, argon laser, krypton laser, xenon laser, nitrogen laser, CO₂ laser, CO laser, argon-fluorine (ArF) excimer laser, krypton-fluorine (KrF) excimer laser, xenon chlorine (XeCl) excimer laser or xenon-fluorine (XeF) excimer laser or a combination thereof. In other instances, the subject flow cytometers include a dye laser, such as a stilbene, coumarin or rhodamine laser. In yet other instances, lasers of interest include a metal-vapor laser, such as a helium-cadmium (HeCd) laser, helium-mercury (HeHg) laser, helium-selenium (HeSe) laser, helium-silver (HeAg) laser, strontium laser, neon-copper (NeCu) laser, copper laser or gold laser and combinations thereof. In still other instances, the subject flow cytometers include a solid-state laser, such as a ruby laser, an Nd:YAG laser, NdCrYAG laser, Er:YAG laser, Nd:YLF laser, Nd:YVO₄ laser, Nd:YCa₄O(BO₃)₃ laser, Nd:YCOB laser, titanium sapphire laser, thulim YAG laser, ytterbium YAG laser, ytterbium₂O₃ laser or cerium doped lasers and combinations thereof.

Aspects of the invention also include a forward scatter detector configured to detect forward scattered light. The number of forward scatter detectors in the subject flow cytometers may vary as desired. For example, the subject flow cytometers may include one forward scatter detector or multiple forward scatter detectors, such as two or more, such as three or more, such as four or more, and including five or more. In certain embodiments, flow cytometers include one forward scatter detector. In other embodiments, flow cytometers include two forward scatter detectors.

Any convenient detector for detecting collected light may be used in the forward scatter detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from, such as from 0.1 cm² to 8 cm², such as from 0.5 cm² to 7 cm² and including from 1 cm² to 5 cm².

Where the subject flow cytometers include multiple forward scatter detectors, each detector may be the same, or the collection of detectors may be a combination of different types of detectors. For example, where the subject flow cytometers include two forward scatter detectors, in some embodiments the first forward scatter detector is a CCD-type device and the second forward scatter detector (or imaging sensor) is a CMOS-type device. In other embodiments, both the first and second forward scatter detectors are CCD-type devices. In yet other embodiments, both the first and second forward scatter detectors are CMOS-type devices. In still other embodiments, the first forward scatter detector is a CCD-type device and the second forward scatter detector is a photomultiplier tube (PMT). In still other embodiments, the first forward scatter detector is a CMOS-type device and the second forward scatter detector is a photomultiplier tube. In yet other embodiments, both the first and second forward scatter detectors are photomultiplier tubes.

In embodiments, the forward scatter detector is configured to measure light continuously or in discrete intervals. In some instances, detectors of interest are configured to take measurements of the collected light continuously. In other instances, detectors of interest are configured to take measurements in discrete intervals, such as measuring light every 0.001 millisecond, every 0.01 millisecond, every 0.1 millisecond, every 1 millisecond, every 10 milliseconds, every 100 milliseconds and including every 1,000 milliseconds, or some other interval.

Embodiments of flow cytometers also include a light dispersion/separator module positioned between the flow cell and the forward scatter detector. Light dispersion devices of interest include but are not limited to, colored glass, bandpass filters, interference filters, dichroic mirrors, diffraction gratings, monochromators and combinations thereof, among other wavelength separating devices. In some embodiments, a bandpass filter is positioned between the flow cell and the forward scatter detector. In other embodiments, more than one bandpass filter is positioned between the flow cell and the forward scatter detector, such as, for example, two or more, three or more, four or more, and including five or more. In embodiments, the bandpass filters have a minimum bandwidths ranging from 2 nm to 100 nm, such as from 3 nm to 95 nm, such as from 5 nm to 95 nm, such as from 10 nm to 90 nm, such as from 12 nm to 85 nm, such as from 15 nm to 80 nm and including bandpass filters having minimum bandwidths ranging from 20 nm to 50 nm wavelengths and reflects light with other wavelengths to the forward scatter detector.

Certain embodiments of flow cytometers include a side scatter detector configured to detect side scatter wavelengths of light (e.g., light refracted and reflected from the surfaces and internal structures of the particle). In other embodiments, flow cytometers include multiple side scatter detectors, such as two or more, such as three or more, such as four or more, and including five or more.

Any convenient detector for detecting collected light may be used in the side scatter detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from, such as from 0.1 cm² to 8 cm², such as from 0.5 cm² to 7 cm² and including from 1 cm² to 5 cm².

Where the subject flow cytometers include multiple side scatter detectors, each side scatter detector may be the same, or the collection of side scatter detectors may be a combination of different types of detectors. For example, where the subject flow cytometers include two side scatter detectors, in some embodiments the first side scatter detector is a CCD-type device and the second side scatter detector (or imaging sensor) is a CMOS-type device. In other embodiments, both the first and second side scatter detectors are CCD-type devices. In yet other embodiments, both the first and second side scatter detectors are CMOS-type devices. In still other embodiments, the first side scatter detector is a CCD-type device, and the second side scatter detector is a photomultiplier tube (PMT). In still other embodiments, the first side scatter detector is a CMOS-type device, and the second side scatter detector is a photomultiplier tube. In yet other embodiments, both the first and second side scatter detectors are photomultiplier tubes.

Embodiments of flow cytometers also include a light dispersion/separator module positioned between the flow cell and the side scatter detector. Light dispersion devices of interest include but are not limited to, colored glass, bandpass filters, interference filters, dichroic mirrors, diffraction gratings, monochromators and combinations thereof, among other wavelength separating devices.

In embodiments, the subject flow cytometers also include a fluorescent light detector configured to detect one or more fluorescent wavelengths of light. In other embodiments, flow cytometers include multiple fluorescent light detectors such as two or more, such as three or more, such as four or more, five or more and including six or more.

Any convenient detector for detecting collected light may be used in the fluorescent light detector described herein. Detectors of interest may include, but are not limited to, optical sensors or detectors, such as active-pixel sensors (APSs), avalanche photodiodes, image sensors, charge-coupled devices (CCDs), intensified charge-coupled devices (ICCDs), light emitting diodes, photon counters, bolometers, pyroelectric detectors, photoresistors, photovoltaic cells, photodiodes, photomultiplier tubes (PMTs), phototransistors, quantum dot photoconductors or photodiodes and combinations thereof, among other detectors. In certain embodiments, the collected light is measured with a charge-coupled device (CCD), semiconductor charge-coupled devices (CCD), active pixel sensors (APS), complementary metal-oxide semiconductor (CMOS) image sensors or N-type metal-oxide semiconductor (NMOS) image sensors. In certain embodiments, the detector is a photomultiplier tube, such as a photomultiplier tube having an active detecting surface area of each region that ranges from 0.01 cm² to 10 cm², such as from 0.05 cm² to 9 cm², such as from, such as from 0.1 cm² to 8 cm², such as from 0.5 cm² to 7 cm² and including from 1 cm² to 5 cm².

Where the subject flow cytometers include multiple fluorescent light detectors, each fluorescent light detector may be the same, or the collection of fluorescent light detectors may be a combination of different types of detectors. For example, where the subject flow cytometers include two fluorescent light detectors, in some embodiments the first fluorescent light detector is a CCD-type device and the second fluorescent light detector (or imaging sensor) is a CMOS-type device. In other embodiments, both the first and second fluorescent light detectors are CCD-type devices. In yet other embodiments, both the first and second fluorescent light detectors are CMOS-type devices. In still other embodiments, the first fluorescent light detector is a CCD-type device and the second fluorescent light detector is a photomultiplier tube (PMT). In still other embodiments, the first fluorescent light detector is a CMOS-type device and the second fluorescent light detector is a photomultiplier tube. In yet other embodiments, both the first and second fluorescent light detectors are photomultiplier tubes.

Embodiments of flow cytometers also include a light dispersion/separator module positioned between the flow cell and the fluorescent light detector. Light dispersion devices of interest include but are not limited to, colored glass, bandpass filters, interference filters, dichroic mirrors, diffraction gratings, monochromators and combinations thereof, among other wavelength separating devices.

In embodiments of flow cytometers, fluorescent light detectors of interest are configured to measure collected light at one or more wavelengths, such as at two or more wavelengths, such as at five or more different wavelengths, such as at ten or more different wavelengths, such as at 25 or more different wavelengths, such as at 50 or more different wavelengths, such as at 100 or more different wavelengths, such as at 200 or more different wavelengths, such as at 300 or more different wavelengths and including measuring light emitted by a sample in the flow stream at 400 or more different wavelengths. In some embodiments, two or more detectors in a flow cytometer as described herein are configured to measure the same or overlapping wavelengths of collected light.

In some embodiments, fluorescent light detectors of interest are configured to measure collected light over a range of wavelengths (e.g., 200 nm - 1000 nm). In certain embodiments, detectors of interest are configured to collect spectra of light over a range of wavelengths. For example, flow cytometers may include one or more detectors configured to collect spectra of light over one or more of the wavelength ranges of 200 nm - 1000 nm. In yet other embodiments, detectors of interest are configured to measure light emitted by a sample in the flow stream at one or more specific wavelengths. For example, flow cytometers may include one or more detectors configured to measure light at one or more of 450 nm, 518 nm, 519 nm, 561 nm, 578 nm, 605 nm, 607 nm, 625 nm, 650 nm, 660 nm, 667 nm, 670 nm, 668 nm, 695 nm, 710 nm, 723 nm, 780 nm, 785 nm, 647 nm, 617 nm and any combinations thereof. In certain embodiments, one or more detectors may be configured to be paired with specific fluorophores, such as those used with the sample in a fluorescence assay.

Suitable flow cytometry systems may include, but are not limited to, those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. Jan;49(pt 1):17-28; Linden, et. al., Semin Throm Hemost. 2004 Oct;30(5):502-11; Alison, et al. J Pathol, 2010 Dec; 222(4):335-344; and Herbig, et al. (2007) Crit Rev Ther Drug Carrier Syst. 24(3):203-255; the disclosures of which are incorporated herein by reference. In certain instances, flow cytometry systems of interest include BD Biosciences FACSCanto™ II flow cytometer, BD Accuri™ flow cytometer, BD Biosciences FACSCelesta™ flow cytometer, BD Biosciences FACSLyric™ flow cytometer, BD Biosciences FACSVerse™ flow cytometer, BD Biosciences FACSymphony™ flow cytometer BD Biosciences LSRFortessa™ flow cytometer, BD Biosciences LSRFortess™ X-20 flow cytometer and BD Biosciences FACSCalibur™ cell sorter, a BD Biosciences FACSCount™ cell sorter, BD Biosciences FACSLyric™ cell sorter and BD Biosciences Via™ cell sorter BD Biosciences Influx™ cell sorter, BD Biosciences Jazz™ cell sorter, BD Biosciences Aria™ cell sorters and BD Biosciences FACSMelody™ cell sorter, or the like. In some instances, the cell sorter is a BD FACSymphony™ S6 cell sorter; BD FACSMelody™ cell sorter; BD FACSAria™ III cell sorter; BD FACSAria™ Fusion cell sorter; BD FACSJazz™ or BD Influx™ cell sorter.

In some embodiments, the subject systems are flow cytometric systems, such those described in U.S. Pat. Nos. 10,663,476; 10,620,111; 10,613,017; 10,605,713; 10,585,031; 10,578,542; 10,578,469; 10,481,074; 10,302,545; 10,145,793; 10,113,967; 10,006,852; 9,952,076; 9,933,341; 9,726,527; 9,453,789; 9,200,334; 9,097,640; 9,095,494; 9,092,034; 8,975,595; 8,753,573; 8,233,146; 8,140,300; 7,544,326; 7,201,875; 7,129,505; 6,821,740; 6,813,017; 6,809,804; 6,372,506; 5,700,692; 5,643,796; 5,627,040; 5,620,842; 5,602,039; 4,987,086; 4,498,766; the disclosures of which are herein incorporated by reference in their entirety.

In some embodiments, particle sorting systems of interest are configured to sort particles, such as cells, with an enclosed particle sorting module, such as those described in U.S. Pat. Publication No. 2017/0299493, filed on Mar. 28, 2017, the disclosure of which is incorporated herein by reference. In certain embodiments, particles (e.g., cells) of the sample are sorted using a sort decision module having a plurality of sort decision units, such as those described in U.S. Provisional Pat. Application No. 16/725,756, filed on Dec. 23, 2019, the disclosure of which is incorporated herein by reference.

In certain instances, the subject particle sorters are flow cytometry systems configured for imaging particles in a flow stream by fluorescence imaging using radiofrequency tagged emission (FIRE), such as those described in Diebold, et al. Nature Photonics Vol. 7(10); 806-810 (2013) as well as described in U.S. Pat. Nos. 9,423,353; 9,784,661; 9,983,132; 10,006,852; 10,078,045; 10,036,699; 10,222,316; 10,288,546; 10,324,019; 10,408,758; 10,451,538; 10,620,111; and U.S. Pat. Publication Nos. 2017/0133857; 2017/0328826; 2017/0350803; 2018/0275042; 2019/0376895 and 2019/0376894 the disclosures of which are herein incorporated by reference.

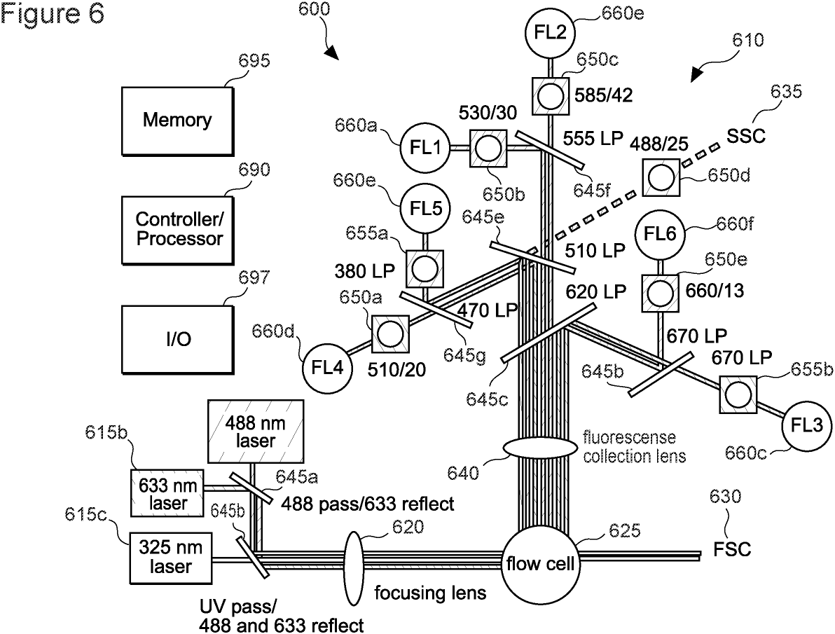

FIG. 6 shows a system 600 for flow cytometry in accordance with an illustrative embodiment of the present invention. The system 600 includes a flow cytometer 610, a controller/processor 690 and a memory 695. The flow cytometer 610 includes one or more excitation lasers 615a-615c, a focusing lens 620, a flow chamber 625, a forward scatter detector 630, a side scatter detector 635, a fluorescence collection lens 640, one or more beam splitters 645a-645g, one or more bandpass filters 650a-650e, one or more longpass (“LP”) filters 655a-655b, and one or more fluorescent light detectors 660a-660f.

The excitation lasers 615a-c emit light in the form of a laser beam. The wavelengths of the laser beams emitted from excitation lasers 615a-615c are 488 nm, 633 nm, and 325 nm, respectively, in the example system of FIG. 6. The laser beams are first directed through one or more of beam splitters 645a and 645b. Beam splitter 645a transmits light at 488 nm and reflects light at 633 nm. Beam splitter 645b transmits UV light (light with a wavelength in the range of 10 to 400 nm) and reflects light at 488 nm and 633 nm.

The laser beams are then directed to a focusing lens 620, which focuses the beams onto the portion of a fluid stream where particles of a sample are located, within the flow chamber 625. The flow chamber is part of a fluidics system which directs particles, typically one at a time, in a stream to the focused laser beam for interrogation. The flow chamber can comprise a flow cell in a benchtop cytometer or a nozzle tip in a stream-in-air cytometer.

The light from the laser beam(s) interacts with the particles in the sample by diffraction, refraction, reflection, scattering, and absorption with re-emission at various different wavelengths depending on the characteristics of the particle such as its size, internal structure, and the presence of one or more fluorescent molecules attached to or naturally present on or in the particle. The fluorescence emissions as well as the diffracted light, refracted light, reflected light, and scattered light may be routed to one or more of the forward scatter detector 630, side scatter detector 635, and the one or more fluorescent light detectors 660a-660f through one or more of the beam splitters 645a-645g, the bandpass filters 650a-650e, the longpass filters 655a-655b, and the fluorescence collection lens 640.

The fluorescence collection lens 640 collects light emitted from the particle- laser beam interaction and routes that light towards one or more beam splitters and filters. Bandpass filters, such as bandpass filters 650a-650e, allow a narrow range of wavelengths to pass through the filter. For example, bandpass filter 650a is a 510/20 filter. The first number represents the center of a spectral band. The second number provides a range of the spectral band. Thus, a 510/20 filter extends 10 nm on each side of the center of the spectral band, or from 500 nm to 520 nm. Shortpass filters transmit wavelengths of light equal to or shorter than a specified wavelength. Longpass filters, such as longpass filters 655a-655b, transmit wavelengths of light equal to or longer than a specified wavelength of light. For example, longpass filter 655a, which is a 670 nm longpass filter, transmits light equal to or longer than 670 nm. Filters are often selected to optimize the specificity of a detector for a particular fluorescent dye. The filters can be configured so that the spectral band of light transmitted to the detector is close to the emission peak of a fluorescent dye.

Beam splitters direct light of different wavelengths in different directions. Beam splitters can be characterized by filter properties such as shortpass and longpass. For example, beam splitter 645g is a 470 LP beam splitter, meaning that the beam splitter 645g transmits wavelengths of light that are 470 nm or longer and reflects wavelengths of light that are shorter than 470 nm in a different direction. In one embodiment, the beam splitters 645a-645g can comprise optical mirrors, such as dichroic mirrors.

The forward scatter detector 630 is positioned off axis from the direct beam through the flow cell and is configured to detect diffracted light, the excitation light that travels through or around the particle in mostly a forward direction. The intensity of the light detected by the forward scatter detector is dependent on the overall size of the particle. The forward scatter detector can include a photodiode. The side scatter detector 635 is configured to detect refracted and reflected light from the surfaces and internal structures of the particle and tends to increase with increasing particle complexity of structure. The fluorescence emissions from fluorescent molecules associated with the particle can be detected by the one or more fluorescent light detectors 660a-660f. The side scatter detector 635 and fluorescent light detectors can include photomultiplier tubes. The signals detected at the forward scatter detector 630, the side scatter detector 635 and the fluorescent detectors can be converted to electronic signals (voltages) by the detectors. This data can provide information about the sample.

In operation, cytometer operation is controlled by a controller/processor 690, and the measurement data from the detectors can be stored in the memory 695 and processed by the controller/processor 690. Although not shown explicitly, the controller/processor 690 is coupled to the detectors to receive the output signals therefrom and may also be coupled to electrical and electromechanical components of the flow cytometer 600 to control the lasers, fluid flow parameters, and the like. Input/output (I/O) capabilities 697 may be provided also in the system. The memory 695, controller/processor 690, and I/O 697 may be entirely provided as an integral part of the flow cytometer 610. In such an embodiment, a display may also form part of the I/O capabilities 697 for presenting experimental data to users of the cytometer 600. Alternatively, some or all of the memory 695 and controller/processor 690 and I/O capabilities may be part of one or more external devices such as a general purpose computer. In some embodiments, some or all of the memory 695 and controller/processor 690 can be in wireless or wired communication with the cytometer 610. The controller/processor 690 in conjunction with the memory 695 and the I/O 697 can be configured to perform various functions related to the preparation and analysis of a flow cytometer experiment.

The system illustrated in FIG. 6 includes six different detectors that detect fluorescent light in six different wavelength bands (which may be referred to herein as a “filter window” for a given detector) as defined by the configuration of filters and/or splitters in the beam path from the flow cell 625 to each detector. Different fluorescent molecules used for a flow cytometer experiment will emit light in their own characteristic wavelength bands. The particular fluorescent labels used for an experiment and their associated fluorescent emission bands may be selected to generally coincide with the filter windows of the detectors. However, as more detectors are provided, and more labels are utilized, perfect correspondence between filter windows and fluorescent emission spectra is not possible. It is generally true that although the peak of the emission spectra of a particular fluorescent molecule may lie within the filter window of one particular detector, some of the emission spectra of that label will also overlap the filter windows of one or more other detectors. This may be referred to as spillover. The I/O 697 can be configured to receive data regarding a flow cytometer experiment having a panel of fluorescent labels and a plurality of cell populations having a plurality of markers, each cell population having a subset of the plurality of markers. The I/O 697 can also be configured to receive biological data assigning one or more markers to one or more cell populations, marker density data, emission spectrum data, data assigning labels to one or more markers, and cytometer configuration data. Flow cytometer experiment data, such as label spectral characteristics and flow cytometer configuration data can also be stored in the memory 695. The controller/processor 690 can be configured to evaluate one or more assignments of labels to markers.

One of skill in the art will recognize that a flow cytometer in accordance with an embodiment of the present invention is not limited to the flow cytometer depicted in FIG. 6, but can include any flow cytometer known in the art. For example, a flow cytometer may have any number of lasers, beam splitters, filters, and detectors at various wavelengths and in various different configurations.

### Processors

In certain embodiments, systems additionally include a processor having memory operably coupled to the processor wherein the memory includes instructions stored thereon, which when executed by the processor, cause the processor to estimate a minimum inhibitory concentration of an antibiotic for a bacterial species based on cytometric data (e.g., flow cytometry data) by computing distance values that reflect a measure of variation between one or more pairs of samples and assigning a minimum inhibitory concentration based on the computed distance values .

In embodiments, after cytometric data (e.g., flow cytometer data) is obtained (e.g., by or from a flow cytometer), the processor is configured to compute distance values that reflect a measure of variation between one or more pairs of samples and assign a minimum inhibitory concentration based on the computed distance values.

In some embodiments, the memory comprises further instructions stored thereon, which, when executed by the processor, cause the processor to assign a minimum inhibitory concentration based on the computed distance values by: fitting a curve to a plot of the distance values of the plurality of samples versus corresponding antibiotic concentrations of the plurality of samples, and assigning a minimum inhibitory concentration based on the fitted curve.

In instances of systems according to the present disclosure, the computed distance values are based on probability binning. In such cases, probability binning may be based on a chi-squared statistic. In embodiments of systems, the memory comprises further instructions stored thereon, which, when executed by the processor, cause the processor to compute distance values based on probability binning by: setting ranges of cytometric data detected from cells in the control sample to a plurality of bins so that nearly equal numbers of cells in the control sample can be assigned to each bin in the plurality of bins, assigning cells in one of the test samples to the plurality of bins based on cytometric data detected from cells in the test sample, and computing a distance between the test sample and the control sample based on the cells in the test sample assigned to each bin. In other instances, the computed distance values are based on a T statistic.

In embodiments of the subject systems, the curve fitted to the plot of the distance values of the plurality of samples versus corresponding antibiotic concentrations of the plurality of samples is a logistic curve. In some examples, the lower horizontal asymptote of the fitted logistic curve is assigned a distance of zero. In some examples, the upper horizontal asymptote of the fitted logistic curve represents concentrations of the antibiotic at which substantially the entire sample is affected by the antibiotic.

In some instances, the memory comprises further instructions stored thereon, which, when executed by the processor, cause the processor to assign a minimum inhibitory concentration to be the antibiotic concentration corresponding to a point at which the slope of the logistic curve is maximum. In other instances, the memory comprises further instructions stored thereon, which, when executed by the processor, cause the processor to assign a minimum inhibitory concentration to be the antibiotic concentration corresponding to a point which is halfway between the upper and lower horizontal asymptotes of the logistic curve. In still other instances, the memory comprises further instructions stored thereon, which, when executed by the processor, cause the processor to assign a minimum inhibitory concentration the antibiotic concentration corresponding to a point that is a reliable detection limit of the curve.

In some embodiments of systems according to the present disclosure, the memory comprises further instructions stored thereon, which, when executed by the processor, cause the processor to compute distance values between one or more pairs of samples by: assigning cells of each sample to clusters of cell populations based on cytometric data from cells in each sample, matching clusters of cell populations from each sample with corresponding clusters of cell populations from one or more other samples, computing distance values between corresponding clusters of cell populations from pairs of samples based on cytometric data from cells in each cluster, and computing distance values between samples based on distance values between corresponding clusters of each sample.

In some cases, the processor is configured to generate one or more population clusters based on the determined parameters of analytes (e.g., cells, particles, nucleic acids) in the sample. In these embodiments, the processor receives cytometric data, calculates parameters of each analyte, and clusters analytes together based on the calculated parameters. For example, where the cytometric data is flow cytometer data, an experiment may include particles labeled by several fluorophores or fluorescently labeled antibodies, and groups of particles may be defined by populations corresponding to one or more fluorescent measurements. In the example, a first group may be defined by a certain range of light scattering for a first fluorophore, and a second group may be defined by a certain range of light scattering for a second fluorophore. If the first and second fluorophores are represented on an x and y axis, respectively, two different color-coded populations might appear to define each group of particles, if the information were to be graphically displayed. Any number of analytes may be assigned to a cluster, including five or more analytes, such as ten or more analytes, such as 50 or more analytes, such as 100 or more analytes, such as 500 analytes and including 1000 analytes. In certain embodiments, the method groups together in a cluster rare events (e.g., rare cells in a sample) detected in the sample. In these embodiments, the analyte clusters generated may include ten or fewer assigned analytes, such as nine or fewer and including five or fewer assigned analytes.

In some examples of systems according to the present disclosure, assigning cells of each sample to clusters of cell populations comprises applying k-means clustering. In other examples, assigning cells of each sample to clusters of cell populations comprises applying a Self-Organizing Map.

In some embodiments of systems of interest, matching corresponding clusters of cell populations from each sample comprises applying a mixed edge cover algorithm.

In some instances of systems according to the present disclosure, computing distances between corresponding clusters is based on distribution parameters of each cluster. In certain instances, computing distances between corresponding clusters comprises measuring a distance between a cluster from a first test sample and a corresponding cluster from each other test sample and the control sample. In some cases, the distance values between corresponding clusters are computed using a Euclidean distance measurement. In other cases, the distance values between corresponding clusters are computed using a Mahalanobis distance measurement.

In some embodiments of systems of interest, the memory comprises further instructions stored thereon, which, when executed by the processor, cause the processor to assign each sample to a branch of a hierarchical tree based on distance values between samples. In other embodiments, the memory comprises further instructions stored thereon, which, when executed by the processor, cause the processor to assign samples to groups based on distances between samples. In such instances, a minimum inhibitory concentration is the antibiotic concentration corresponding to the sample with the lowest antibiotic concentration in a first group of samples that is the furthest distance away from a second group of samples, wherein the second group of samples includes the untreated control sample.

In certain embodiments of systems according to the present disclosure, a susceptibility or resistance of the antibiotic for the bacterial species is determined based on the minimum inhibitory concentration.

In instances of systems of interest, the cytometric data is multi-parametric cytometry data. In such instances, the cytometric data may comprise light scatter or marker data or a combination thereof. In some examples, the marker data comprises fluorescent light emission data. In other examples, the fluorescent light emission data comprises frequency-encoded fluorescence data from cells.

In some embodiments of systems, the apparatus is configured to obtain the cytometric data by analyzing the plurality of test samples and the control sample for the antibiotic and bacterial species. In other embodiments, the apparatus is configured to obtain cytometric data from the plurality of test samples and the control sample by flow cytometrically analyzing the plurality of test samples and control sample.

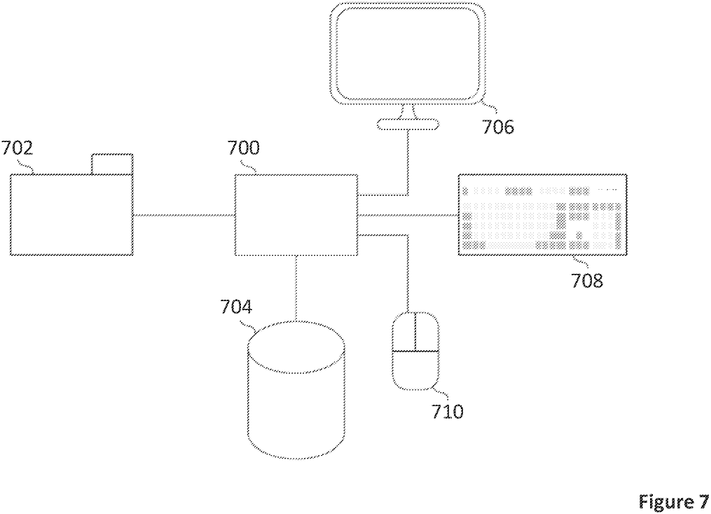

FIG. 7 shows a functional block diagram for one example of a processor 700, for analyzing and displaying data. A processor 700 can be configured to implement a variety of processes for controlling graphic display of biological events.

An apparatus 702 can be configured to obtain cytometric data. In some cases, the apparatus can be configured to obtain the cytometric data by analyzing the plurality of test samples and the control sample for the antibiotic and bacterial species. In some embodiments, the apparatus is configured to obtain cytometric data from the plurality of test samples and the control sample by flow cytometrically analyzing the plurality of test samples and control sample. That is, in embodiments, the apparatus may be, or may be operably connected to, a flow cytometer (e.g., as described above). For example, a flow cytometer can generate cytometric data that is flow cytometer data. The apparatus can be configured to provide biological event data to the processor 700. A data communication channel can be included between the apparatus 702 and the processor 700. The data can be provided to the processor 700 via the data communication channel. In embodiments where the apparatus is a flow cytometer, data received from the apparatus 702 includes cytometric data that is flow cytometer data. The processor 700 can be configured to provide a graphical display including plots (e.g., such as those shown in FIG. 3 or FIG. 5, as described above) to display 706. The processor 700 can be further configured to render a gate around populations of data shown by the display device 706, overlaid upon the plot, for example. In some embodiments, the gate can be a logical combination of one or more graphical regions of interest drawn upon a single parameter histogram or bivariate plot. In some embodiments, the display can be used to display analyte parameters or saturated detector data.

The processor 700 can be further configured to display data on the display device 706 within the gate differently from other events in the biological event data outside of the gate. For example, the processor 700 can be configured to render the color of biological event data contained within the gate to be distinct from the color of biological event data outside of the gate. In this way, the processor 700 may be configured to render different colors to represent each unique population of data. The display device 706 can be implemented as a monitor, a tablet computer, a smartphone, or other electronic device configured to present graphical interfaces.

The processor 700 can be configured to receive a gate selection signal identifying the gate from a first input device. For example, the first input device can be implemented as a mouse 710. The mouse 710 can initiate a gate selection signal to the processor 700 identifying the population to be displayed on or manipulated via the display device 706 (e.g., by clicking on or in the desired gate when the cursor is positioned there). In some implementations, the first device can be implemented as the keyboard 708 or other means for providing an input signal to the processor 700 such as a touchscreen, a stylus, an optical detector, or a voice recognition system. Some input devices can include multiple inputting functions. In such implementations, the inputting functions can each be considered an input device. For example, as shown in FIG. 7, the mouse 710 can include a right mouse button and a left mouse button, each of which can generate a triggering event.

The triggering event can cause the processor 700 to alter the manner in which the data is displayed, which portions of the data is actually displayed on the display device 706, and/or provide input to further processing such as selection of a population of interest for analysis.

In some embodiments, the processor 700 can be configured to detect when gate selection is initiated by the mouse 710. The processor 700 can be further configured to automatically modify plot visualization to facilitate the gating process. The modification can be based on the specific distribution of data received by the processor 700.

The processor 700 can be connected to a storage device 704. The storage device 704 can be configured to receive and store data from the processor 700. The storage device 704 can be further configured to allow retrieval of data, such as cytometric data consisting of flow cytometric event data, by the processor 700.

A display device 706 can be configured to receive display data from the processor 700. The display data can comprise plots of biological event data and gates outlining sections of the plots. The display device 706 can be further configured to alter the information presented according to input received from the processor 700 in conjunction with input from apparatus 702, the storage device 704, the keyboard 708, and/or the mouse 710.

In some implementations the processor 700 can generate a user interface to receive example events for sorting. For example, the user interface can include a control for receiving example events or example images. The example events or images or an example gate can be provided prior to obtaining cytometric data, such as via collection of event data for a sample, or based on an initial set of events for a portion of the sample.

### Computer-Controlled Systems

Aspects of the present disclosure further include computer-controlled systems. where the systems further include one or more computers for complete automation or partial automation. In some embodiments, systems include a computer having a computer readable storage medium with a computer program stored thereon, where the computer program when loaded on the computer includes instructions for estimating a minimum inhibitory concentration of an antibiotic for a bacterial species. In embodiments, the computer program when loaded on the computer includes instructions for computing distance values that reflect a measure of variation between one or more pairs of samples, and assigning a minimum inhibitory concentration based on the computed distance values. In some embodiments, the computer program when loaded on the computer includes instructions for fitting a curve to a plot of the distance values of the plurality of samples versus corresponding antibiotic concentrations of the plurality of samples, and assigning a minimum inhibitory concentration based on the fitted curve. In other embodiments, the computer program when loaded on the computer includes instructions for computing distance values between one or more pairs of samples by: assigning cells of each sample to clusters of cell populations based on cytometric data from cells in each sample, matching clusters of cell populations from each sample with corresponding clusters of cell populations from one or more other samples, computing distance values between corresponding clusters of cell populations from pairs of samples based on cytometric data from cells in each cluster, and computing distance values between samples based on distance values between corresponding clusters of each sample. Such embodiments may further comprise instructions for assigning samples to groups based on distances between samples. In still other embodiments, a susceptibility or resistance of the antibiotic for the bacterial species is determined based on the minimum inhibitory concentration.

In embodiments, the system is configured to analyze the data within a software or an analysis tool for analyzing flow cytometer data, such as FlowJo®. FlowJo® is a software package developed by FlowJo LLC (a subsidiary of Becton Dickinson) for analyzing flow cytometer data. The software is configured to manage flow cytometer data and produce graphical reports thereon (https://www(dot)flowjo(dot)com/learn/flowjo-university/flowjo). The initial data can be analyzed within the data analysis software or tool (e.g., FlowJo®) by appropriate means, such as manual gating, cluster analysis, or other computational techniques. The instant systems, or a portion thereof, can be implemented as software components of a software for analyzing data, such as FlowJo®. In these embodiments, computer-controlled systems according to the instant disclosure may function as a software “plugin” for an existing software package, such as FlowJo®.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor, or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, Python, C, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, processors include analog electronics which allows the user to manually align a light source with the flow stream based on the first and second light signals. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described comprising a computer usable medium having control logic (computer software program, including program code) stored therein. The control logic, when executed by the processor of the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to the processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician’s office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a work station, or other computer type. They may be connected via any known or future type of cabling or other communication system including wireless systems, either networked or otherwise. They may be co-located, or they may be physically separated. Various operating systems may be employed on any of the computer platforms, possibly depending on the type and/or make of computer platform chosen. Appropriate operating systems include Windows NT, Windows XP, Windows 7, Windows 8, iOS, Sun Solaris, Linux, OS/400, Compaq Tru64 Unix, SGI IRIX, Siemens Reliant Unix, and others.

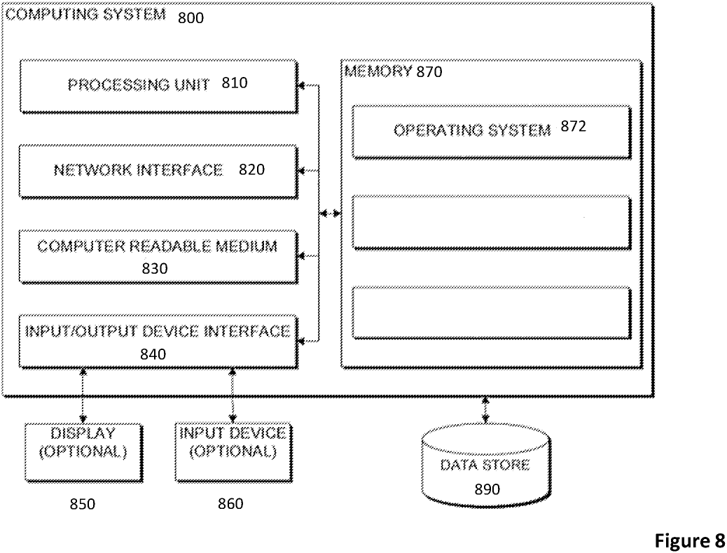

FIG. 8 depicts a general architecture of an example computing device 800 according to certain embodiments. The general architecture of the computing device 800 depicted in FIG. 8 includes an arrangement of computer hardware and software components. It is not necessary, however, that all of these generally conventional elements be shown in order to provide an enabling disclosure. As illustrated, the computing device 800 includes a processing unit 810, a network interface 820, a computer readable medium drive 830, an input/output device interface 840, a display 850, and an input device 860, all of which may communicate with one another by way of a communication bus. The network interface 820 may provide connectivity to one or more networks or computing systems. The processing unit 810 may thus receive information and instructions from other computing systems or services via a network. The processing unit 810 may also communicate to and from memory 870 and further provide output information for an optional display 850 via the input/output device interface 840. For example, an analysis software (e.g., data analysis software or program such as FlowJo®) stored as executable instructions in the non-transitory memory of the analysis system can display the cytometric data, such as flow cytometry event data, to a user. The input/output device interface 840 may also accept input from the optional input device 860, such as a keyboard, mouse, digital pen, microphone, touch screen, gesture recognition system, voice recognition system, gamepad, accelerometer, gyroscope, or other input device.

The memory 870 may contain computer program instructions (grouped as modules or components in some embodiments) that the processing unit 810 executes in order to implement one or more embodiments. The memory 870 generally includes RAM, ROM and/or other persistent, auxiliary or non-transitory computer-readable media. The memory 870 may store an operating system 872 that provides computer program instructions for use by the processing unit 810 in the general administration and operation of the computing device 800. Data may be stored in data storage device 890. The memory 870 may further include computer program instructions and other information for implementing aspects of the present disclosure.

### Computer-Readable Storage Medium

Aspects of the present disclosure further include non-transitory computer readable storage media having instructions for practicing the subject methods. Computer readable storage media may be employed on one or more computers for complete automation or partial automation of a system for practicing methods described herein. In some embodiments, instructions in accordance with the method described herein can be coded onto a computer-readable medium in the form of “programming,” where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to a computer. In some instances, instructions may be provided on an integrated circuit device. Integrated circuit devices of interest may include, in certain instances, a reconfigurable field programmable gate array (FPGA), an application specific integrated circuit (ASIC) or a complex programmable logic device (CPLD). A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Java (Sun Microsystems, Inc., Santa Clara, CA), Visual Basic (Microsoft Corp., Redmond, WA), Perl, Python, C, and C++ (AT&T Corp., Bedminster, NJ), as well as any many others.

In some embodiments, computer readable storage media of interest include a computer program stored thereon, where the computer program when loaded on a computer includes instructions estimating a minimum inhibitory concentration of an antibiotic for a bacterial species. Specifically, computer readable storage media of interest include instructions comprising algorithm for obtaining cytometric data for a plurality of test samples and a control sample for the antibiotic and bacterial species, algorithm for computing distance values that reflect a measure of variation between one or more pairs of samples, and algorithm for assigning a minimum inhibitory concentration based on the computed distance values.

In some embodiments, computer readable storage media of interest include instructions for assigning a minimum inhibitory concentration based on the computed distance values by, fitting a curve to a plot of the distance values of the plurality of samples versus corresponding antibiotic concentrations of the plurality of samples, and assigning a minimum inhibitory concentration based on the fitted curve. In other embodiments, computer readable storage media of interest include instructions for computing distance values based on probability binning by setting ranges of cytometric data detected from cells in the control sample to a plurality of bins so that nearly equal numbers of cells in the control sample can be assigned to each bin in the plurality of bins, assigning cells in one of the test samples to the plurality of bins based on cytometric data detected from cells in the test sample, and computing a distance between the test sample and the control sample based on the cells in the test sample assigned to each bin.

In some embodiments, computer readable storage media of interest include instructions for computing distance values between one or more pairs of samples by: assigning cells of each sample to clusters of cell populations based on cytometric data from cells in each sample, matching clusters of cell populations from each sample with corresponding clusters of cell populations from one or more other samples, computing distance values between corresponding clusters of cell populations from pairs of samples based on cytometric data from cells in each cluster, and computing distance values between samples based on distance values between corresponding clusters of each sample. In such embodiments, computer readable storage media of interest may include instructions for assigning each sample to a branch of a hierarchical tree based on distance values between samples and/or for assigning samples to groups based on distances between samples.

In embodiments, the system is configured to process and/or analyze data within a software or an analysis tool for analyzing cytometric data, such as flow cytometer data, such as FlowJo®. The data can be analyzed within the data analysis software or tool (e.g., FlowJo®) by appropriate means, such as manual gating, cluster analysis, or other computational techniques. The instant systems, or a portion thereof, can be implemented as software components of a software for processing and/or analyzing data, such as FlowJo®. In these embodiments, computer-controlled systems according to the instant disclosure may function as a software “plugin” for an existing software package, such as FlowJo®.

The computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor, or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Visual Basic, Perl, Python, C, C++ or other high level or low level languages, as well as combinations thereof, as is known in the art. The operating system also provides scheduling, input-output control, file and data management, memory management, and communication control and related services, all in accordance with known techniques.

### Utility

The subject methods, systems and non-transitory computer readable storage media find use in a variety of applications where it is desirable to estimate a minimum inhibitory concentration of an antibiotic for a bacterial species. For example, the present disclosure can be employed to characterize many types of antibiotic and bacterial species combinations, in particular, antibiotic and bacterial species combinations relevant to medical treatment or protocols for caring for a patient. The present disclosure can be employed to estimate the effectiveness of an antibiotic with respect to a bacterial species in an objective and/or automatic way. Embodiments of the invention facilitate making reproduceable estimates of a minimum inhibitory concentration of an antibiotic with respect to a bacterial species. Embodiments of the invention also facilitate making reproduceable estimates of a susceptibility or resistance of an antibiotic with respect to a bacterial species. Further, samples can be from in vitro or in vivo sources.

Although the foregoing invention has been described in some detail by way of illustration and example for purposes of clarity of understanding, it is readily apparent to those of ordinary skill in the art in light of the teachings of this invention that some changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of estimating a minimum inhibitory concentration of an antibiotic for a bacterial species, the method comprising:
obtaining cytometric data for a plurality of test samples and a control sample for the antibiotic and bacterial species;
computing distance values that reflect a measure of variation between one or more pairs of samples; and
assigning a minimum inhibitory concentration based on the computed distance values.

2. The method according to claim 1, wherein assigning a minimum inhibitory concentration based on the computed distance values comprises:
fitting a curve to a plot of the distance values of the plurality of samples versus corresponding antibiotic concentrations of the plurality of samples; and
assigning a minimum inhibitory concentration based on the fitted curve.

3. The method according to claim 2, wherein the computed distance values are based on probability binning.

4. The method according to claim 3, wherein the probability binning is based on a chi-squared statistic.

5. The method according to claim 3, wherein the computed distance values based on probability binning comprise:
setting ranges of cytometric data detected from cells in the control sample to a plurality of bins so that nearly equal numbers of cells in the control sample can be assigned to each bin in the plurality of bins;
assigning cells in one of the test samples to the plurality of bins based on cytometric data detected from cells in the test sample; and
computing a distance between the test sample and the control sample based on the cells in the test sample assigned to each bin.

6. The method according to claim 2, wherein the computed distance values are based on a T statistic.

7. The method according to claim 2, wherein the curve fitted to the plot of the distance values of the plurality of samples versus corresponding antibiotic concentrations of the plurality of samples is a logistic curve.

8-12. (canceled)

13. The method according to claim 1, wherein computing distance values between one or more pairs of samples comprises:
assigning cells of each sample to clusters of cell populations based on cytometric data from cells in each sample;
matching clusters of cell populations from each sample with corresponding clusters of cell populations from one or more other samples;
computing distance values between corresponding clusters of cell populations from pairs of samples based on cytometric data from cells in each cluster; and
computing distance values between samples based on distance values between corresponding clusters of each sample.

14-15. (canceled)

16. The method according to claim 13, wherein matching corresponding clusters of cell populations from each sample comprises applying a mixed edge cover algorithm.

17. The method according to claim 13, wherein computing distances between corresponding clusters is based on distribution parameters of each cluster.

18. The method according to claim 13, wherein computing distances between corresponding clusters comprises measuring a distance between a cluster from a first test sample and a corresponding cluster from other test samples and the control sample.

19. The method according to claim 13, wherein the distance values between corresponding clusters are computed using a Euclidean distance measurement.

20. The method according to claim 13, wherein the distance values between corresponding clusters are computed using a Mahalanobis distance measurement.

21. The method according to claim 13, further comprising assigning each sample to a branch of a hierarchical tree based on distance values between samples.

22. The method according to claim 13, further comprising assigning samples to groups based on distances between samples.

23. (canceled)

24. The method according to claim 1, wherein a susceptibility or resistance of the antibiotic for the bacterial species is determined based on the minimum inhibitory concentration.

25. The method according to claim 1, further comprising preparing the plurality of test samples and the control sample.

26. (canceled)

27. The method according to claim 1, wherein the cytometric data is multi-parametric cytometry data.

28. The method according to claim 1, wherein the cytometric data comprises light scatter or marker data or a combination thereof.

29-31. (canceled)

32. The method according to claim 1, wherein obtaining cytometric data from the plurality of test samples and the control sample comprises flow cytometrically analyzing the plurality of test samples and control sample.

33-92. (canceled)
